(19) United States
(12) Patent Application Publication
(10) Pub. No.: US 2018/0221442 A1
(43) Pub. Date: Aug. 9, 2018

(54) INHIBITION OF BONE LOSS AND INHIBITION OF ARTHRITIS BY CTHRC1

(71) Applicant: Maine Medical Center, Scarborough, ME (US)

(72) Inventors: Volkhard Lindner, South Portland, ME (US); Yong-Ri Jin, Scarborough, ME (US)

(21) Appl. No.: 15/877,875

(22) Filed: Jan. 23, 2018

Related U.S. Application Data
(60) Provisional application No. 62/449,481, filed on Jan. 23, 2017.

(51) Int. Cl. A61K 38/17; A61P 19/02; A61P 19/10

(52) U.S. Cl. CPC A61K 38/1709; A61P 19/10; A61P 19/02

(57) ABSTRACT

The invention features compositions and methods for inhibiting bone loss and arthritis.

### CROSS-REFERENCE TO RELATED APPLICATION

This application claims the benefit of priority to U.S. Provisional Patent Application No. 62/449,481, filed in the United States Patent and Trademark Office on Jan. 23, 2017, the entire contents of which are incorporated herein by reference.

### STATEMENT OF RIGHTS TO INVENTIONS MADE UNDER FEDERALLY SPONSORED RESEARCH

This work was supported by The National Institutes of Health under grant number P30GM103392, under grant number P30GM103465, and under grant number P50-HD28934. The government has certain rights in the invention.

### INCORPORATION-BY-REFERENCE OF SEQUENCE LISTING

The content of the text file named “48420_515001US_Sequence_Listing_ST25.txt”, which was created on Jan. 23, 2018, and is 9,619 bytes in size, is hereby incorporated by reference in its entirety.

### FIELD OF THE INVENTION

This invention relates generally to the field of bone loss and arthritis.

### BACKGROUND OF THE INVENTION

CTHRC1 is a circulating factor expressed at sites of tissue injury and remodeling. Prior to the invention described herein, the role of CTHRC1 funtion in bone homeostasis had not been identified. As such, there is a pressing need to identify the the role of CTHRC1 in osteoclast differentiation and the regulation of bone mass.

### SUMMARY OF THE INVENTION

The invention is based, at least in part, on the surprising discovery that CTHRC1 inhibits inflammation and osteoclast function and activity. Specifically, CTHRC1 inhibits osteoclast differentiation and collagen antibody-induced arthritis. Described herein is the identification that Cthrc1 inhibits activation of a signaling pathway that is critical for mediating inflammatory responses. With inflammation, whether acute or chronic, being at the center of many illnesses, Cthrc1 has many therapeutic applications. As described herein, inflammation and bone erosion is much reduced in an arthritis model in the presence of Cthrc1. Accordingly, increasing levels of Cthrc1 in humans is useful for inhibiting inflammation, arthritis, and bone loss. For example, recombinant Cthrc1 is administered to inhibit inflammation, arthritis, and/or bone loss.

A method of treating or preventing bone loss, low bone mass or a low bone mass-associated condition in a subject are carried out by identifying a subject having or at risk of developing low bone mass or a low bone mass-associated condition inhibiting osteoclast differentiation in the subject, thereby treating or preventing bone loss, low bone mass or a low bone mass-associated condition in the subject. Preferably, osteoclast differentiation in the subject is inhibited by administering to the subject an effective amount of a Cthrc1 polypeptide or a Cthrc1 receptor agonist. For example, the low bone mass or low bone mass-associated condition comprises osteoporosis or arthritis. The composition is administered orally, intravenously, intramuscularly, or systemically. The effective amount of CTHRC1 polypeptide is sufficient to inhibit bone loss by at least 5%, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%. In one aspect, the effective amount of CTHRC1 polypeptide is sufficient to increase bone mass by at least 5%, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%. The composition is administered orally, intravenously, intramuscularly, or systemically.

Also provided are methods of treating or preventing inflammation or an inflammation-associated condition in a subject by identifying a subject having or at risk of developing inflammation or an inflammation-associated condition and administering to the subject an effective amount of a Cthrc1 polypeptide or a Cthrc1 receptor agonist, thereby treating or preventing inflammation or an inflammation-associated condition in a subject.

Also provided are methods of inhibiting osteoclast differentiation and activity comprising administering to the subject an effective amount of a Cthrc1 polypeptide or a Cthrc1 receptor agonist, thereby inhibiting osteoclast differentiation and activity. For example, osteoclast differentiation and/or activity is decreased by at least 5%, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

Exemplary effective doses of CTHRC1 polypeptide or a CTHRC1 receptor agonist include between 0.1 μg/kg and 100 mg/kg body weight, e.g., 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 600, 700, 800, or 900 μg/kg body weight or 1, 2, 3, 4, 5, 6, 7, 8, 9, 10, 20, 30, 40, 50, 60, 70, 80, 90, or 100 mg/kg body weight.

In some cases, the CTHRC1 polypeptide or a CTHRC1 receptor agonist is administered daily, e.g., every 24 hours. Or, the CTHRC1 polypeptide or a CTHRC1 receptor agonist is administered continuously or several times per day, e.g., every 1 hour, every 2 hours, every 3 hours, every 4 hours, every 5 hours, every 6 hours, every 7 hours, every 8 hours, every 9 hours, every 10 hours, every 11 hours, or every 12 hours.

Exemplary effective daily doses of CTHRC1 polypeptide or a CTHRC1 receptor agonist include between 0.1 μg/kg and 100 μg/kg body weight, e.g., 0.1, 0.3, 0.5, 1, 5, 10, 15, 20, 25, 30, 35, 40, 45, 50, 55, 60, 65, 70, 75, 80, 85, 90, 95, or 99 μg/kg body weight.

Alternatively, the CTHRC1 polypeptide or a CTHRC1 receptor agonist is administered about once per week, e.g., about once every 7 days. Or, the CTHRC1 polypeptide or a CTHRC1 receptor agonist is administered twice per week, three times per week, four times per week, five times per week, six times per week, or seven times per week. Exemplary effective weekly doses of CTHRC1 polypeptide or a CTHRC1 receptor agonist include between 0.0001 mg/kg and 4 mg/kg body weight, e.g., 0.001, 0.003, 0.005, 0.01. 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, or 4 mg/kg body weight. For example, an effective weekly dose of CTHRC1 polypeptide or a CTHRC1 receptor agonist is between 0.1 μg/kg body weight and 400 μg/kg body weight. Ultimately, the attending physician or veterinarian decides the appropriate amount and dosage regimen.

The subject is preferably a mammal in need of such treatment, e.g., a subject that has been diagnosed with diabetes or a predisposition thereto. The mammal is any mammal, e.g., a human, a primate, a mouse, a rat, a dog, a cat, a horse, as well as livestock or animals grown for food consumption, e.g., cattle, sheep, pigs, chickens, and goats. In a preferred embodiment, the mammal is a human.

Exemplary effective doses of CTHRC1 polypeptide antagonist or a CTHRC1 receptor antagonist include between 0.1 μg/kg and 100 mg/kg body weight, e.g., 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 600, 700, 800, or 900 μg/kg body weight or 1, 2, 3, 4, 5, 6, 7, 8, 9, 10, 20, 30, 40, 50, 60, 70, 80, 90, or 100 mg/kg body weight.

In some cases, the CTHRC1 polypeptide antagonist or a CTHRC1 receptor antagonist is administered daily, e.g., every 24 hours. Or, the CTHRC1 polypeptide antagonist or a CTHRC1 receptor antagonist is administered continuously or several times per day, e.g., every 1 hour, every 2 hours, every 3 hours, every 4 hours, every 5 hours, every 6 hours, every 7 hours, every 8 hours, every 9 hours, every 10 hours, every 11 hours, or every 12 hours.

Exemplary effective daily doses of CTHRC1 polypeptide antagonist or a CTHRC1 receptor antagonist include between 0.1 μg/kg and 100 μg/kg body weight, e.g., 0.1, 0.3, 0.5, 1, 5, 10, 15, 20, 25, 30, 35, 40, 45, 50, 55, 60, 65, 70, 75, 80, 85, 90, 95, or 99 μg/kg body weight.

Alternatively, the CTHRC1 polypeptide antagonist or a CTHRC1 receptor antagonist is administered about once per week, e.g., about once every 7 days. Or, the CTHRC1 polypeptide antagonist or a CTHRC1 receptor antagonist is administered twice per week, three times per week, four times per week, five times per week, six times per week, or seven times per week. Exemplary effective weekly doses of CTHRC1 polypeptide antagonist or a CTHRC1 receptor antagonist include between 0.0001 mg/kg and 4 mg/kg body weight, e.g., 0.001, 0.003, 0.005, 0.01. 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, or 4 mg/kg body weight. For example, an effective weekly dose of CTHRC1 polypeptide antagonist or a CTHRC1 receptor antagonist is between 0.1 μg/kg body weight and 400 μg/kg body weight.

Ultimately, the attending physician or veterinarian decides the appropriate amount and dosage regimen.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

Provided herein are CTHRC1 polypeptide agonists and antagonists.

The term “agonist” as used herein, refers to any molecule which enhances the biological activity of its target molecule.

As used herein, the terms “antagonist” and “inhibitor” are used interchangeably to refer to any molecule that counteracts or inhibits, decreases, or suppresses the biological activity of its target molecule. Suitable CTHRC1 polypeptide antagonists include soluble receptors (e.g., soluble CTHRC1 receptor), peptide inhibitors, small molecule inhibitors, ligand fusions, and antibodies.

The term “receptor antagonist,” as used herein, refers to an agent that is capable of specifically binding and inhibiting signaling through a receptor to fully block or detectably inhibit a response mediated by the receptor.

The agonists or antagonists may include but are not limited to nucleic acids, peptides, antibodies, or small molecules that bind to their specified target or the target's natural ligand and modulate the biological activity.

Provided herein are methods for screening CTHRC1 polypeptide agonists and antagonists, as well as CTHRC1 receptor agonists and antagonists for desired biological activity. For example, a CTHRC1 polypeptide agonist is screened to confirm it enhances the biological activity of a CTHRC1 polypeptide, while a CTHRC1 receptor agonist is screened to confirm that it enhances the biological activity of a CTHRC1 receptor. Similarly, a CTHRC1 polypeptide antagonist is screened to confirm that it counteracts or inhibits, decreases, or suppresses the biological activity of a CTHRC1 polypeptide, while a CTHRC1 receptor antagonist is screened to confirm that it counteracts or inhibits, decreases, or suppresses the biological activity of a CTHRC1 receptor.

In some cases, nucleic acids, e.g., ribonucleic acid (RNA) or deoxyribonucleic acid (DNA), inhibit the expression of CTHRC1 polypeptide or CTHRC1 receptor, thereby inhibiting the activity of CTHRC1 or CTHRC1 receptor. In some cases, the nucleic acid comprises small interfering RNA (siRNA), RNA interference (RNAi), messenger RNA (mRNA), short hairpin RNA (shRNA), or microRNA. Thus, suitable CTHRC1 antagonists include CTHRC1 siRNA and CTHRC1 shRNA, each of which is available from Santa Cruz Biotechnology, Inc., Dallas, Tex. and incorporated herein by reference.

For example, provided herein are small molecule agonists and small molecule antagonists, i.e., inhibitors. A small molecule is a compound that is less than 2000 Daltons in mass. The molecular mass of the small molecule is preferably less than 1000 Daltons, more preferably less than 600 Daltons, e.g., the compound is less than 500 Daltons, less than 400 Daltons, less than 300 Daltons, less than 200 Daltons, or less than 100 Daltons. Small molecules are organic or inorganic. Exemplary organic small molecules include, but are not limited to, aliphatic hydrocarbons, alcohols, aldehydes, ketones, organic acids, esters, mono- and disaccharides, aromatic hydrocarbons, amino acids, and lipids. Exemplary inorganic small molecules comprise trace minerals, ions, free radicals, and metabolites. Alternatively, small molecules can be synthetically engineered to consist of a fragment, or small portion, or a longer amino acid chain to fill a binding pocket of an enzyme. Typically, small molecules are less than one kiloDalton.

Described herein are anti-CTHRC1 antibodies. For example, monoclonal antibodies 10G07 (Duarte et al., 2014 PLOS ONE, 9(6): e100449, incorporated herein by reference), 13D11, and 19C07 are specific for the N terminus of human CTHRC1 and do not react with rat or murine CTHRC1. Anti-CTHRC1 antibody, clone 13E09, recognizes an epitope located within the N terminal half of the molecule of both human and rodent CTHRC1. Also provided is anti-CTHRC1 antibody, H-213, incorporated herein by reference and anti-CTHRC1 antibody, T-19, which is incorporated herein by reference (Santa Cruz Biotechnology, Inc., Dallas, Tex.). Also included are the following anti-CTHRC1 antibodies: SAB1102667, HPA059806, SAB2107469, and SAB1402656, each of which is incorporated herein by reference (Sigma-Aldrich®, St. Louis, Mo.). Also included is the following anti-CTHRC1 antibody PA5-38054, incorporated herein by reference (Thermo Scientific, Waltham, Mass.). In some cases, the anti-CTHRC1 antibodies described herein are administered at a concentration of 0.1 μg/ml to 500 mg/ml.

Also provided are anti-CTHRC1 antibodies, i.e., anti-GPR180 antibodies. For example, provided herein are the following anti-GPR180 antibodies: HPA047250, SAB4500617, SAB1303667, SAB1408931, each of which is incorporated herein by reference (Sigma-Aldrich®, St. Louis, Mo.). Also described herein is the following anti-GRP180 antibody: PA5-26788, incorporated herein by reference (Thermo Scientific, Waltham, Mass.). Also provided is an anti-GPR180 antibody, NBP2-14068, incorporated herein by reference (Novus Biologicals, Littleton, Colo.). Described herein is an anti-GPR180 antibody, AB1N952608, incorporated herein by reference (antibodies-online.com; Atlanta, Ga.). In some cases, the anti-GPR180 antibodies described herein are administered at a concentration of 0.1 μg/ml to 500 mg/ml.

Antibodies and fragments thereof described herein include, but are not limited to, polyclonal, monoclonal, chimeric, dAb (domain antibody), single chain, Fab, Fab′ and F(ab′)2 fragments, Fv, scFvs. A fragment of an antibody possess the immunological activity of its respective antibody. In some embodiments, a fragment of an antibody contains 1500 or less, 1250 of less, 1000 or less, 900 or less, 800 or less, 700 or less, 600 or less, 500 or less, 400 or less, 300 or less, 200 or less amino acids. For example, a protein or peptide inhibitor contains 1500 or less, 1250 of less, 1000 or less, 900 or less, 800 or less, 700 or less, 600 or less, 500 or less, 400 or less, 300 or less, 200 or less, 100 or less, 80 or less, 70 or less, 60 or less, 50 or less, 40 or less, 30 or less, 25 or less, 20 or less, 10 or less amino acids. For example, a nucleic acid inhibitor of the invention contains 400 or less, 300 or less, 200 or less, 150 or less, 100 or less, 90 or less, 80 or less, 70 or less, 60 or less, 50 or less, 40 or less, 35 or less, 30 or less, 28 or less, 26 or less, 24 or less, 22 or less, 20 or less, 18 or less, 16 or less, 14 or less, 12 or less, 10 or less nucleotides.

The term “antibody” (Ab) as used herein includes monoclonal antibodies, polyclonal antibodies, multispecific antibodies (e.g., bispecific antibodies), and antibody fragments, so long as they exhibit the desired biological activity. The term “immunoglobulin” (Ig) is used interchangeably with “antibody” herein.

An “isolated antibody” is one that has been separated and/or recovered from a component of its natural environment. Contaminant components of its natural environment are materials that would interfere with diagnostic or therapeutic uses for the antibody, and may include enzymes, hormones, and other proteinaceous or nonproteinaceous solutes. In preferred embodiments, the antibody is purified: (1) to greater than 95% by weight of antibody as determined by the Lowry method, and most preferably more than 99% by weight; (2) to a degree sufficient to obtain at least 15 residues of N-terminal or internal amino acid sequence by use of a spinning cup sequenator; or (3) to homogeneity by SDS-PAGE under reducing or non-reducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The basic four-chain antibody unit is a heterotetrameric glycoprotein composed of two identical light (L) chains and two identical heavy (H) chains. An IgM antibody consists of 5 of the basic heterotetramer unit along with an additional polypeptide called J chain, and therefore contain 10 antigen binding sites, while secreted IgA antibodies can polymerize to form polyvalent assemblages comprising 2-5 of the basic 4-chain units along with J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (V_{H}) followed by three constant domains (C_{H}) for each of the a and y chains and four C_{H} domains for μ and ε isotypes. Each L chain has at the N-terminus, a variable domain (V_{L}) followed by a constant domain (C_{L}) at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71, and Chapter 6.

The L chain from any vertebrate species can be assigned to one of two clearly distinct types, called kappa (κ) and lambda (γ), based on the amino acid sequences of their constant domains (C_{L}). Depending on the amino acid sequence of the constant domain of their heavy chains (C_{H}), immunoglobulins can be assigned to different classes or isotypes. There are five classes of immunoglobulins: IgA, IgD, IgE, IgG, and IgM, having heavy chains designated alpha (α), delta (δ), epsilon (ε), gamma (γ) and mu (μ), respectively. The γ and α classes are further divided into subclasses on the basis of relatively minor differences in C_{H} sequence and function, e.g., humans express the following subclasses: IgG1, IgG2, IgG3, IgG4, IgA1, and IgA2.

The term “variable” refers to the fact that certain segments of the V domains differ extensively in sequence among antibodies. The V domain mediates antigen binding and defines specificity of a particular antibody for its particular antigen. However, the variability is not evenly distributed across the 110-amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody that are responsible for antigen binding. The hypervariable region generally comprises amino acid residues from a “complementarity determining region” or “CDR” (e.g., around about residues 24-34 (L1), 50-56 (L2) and 89-97 (L3) in the VL, and around about 31-35 (H1), 50-65 (H2) and 95-102 (H3) in the V_{H} when numbered in accordance with the Kabat numbering system; Kabat et al., Sequences of Proteins of Immunological Interest, 5th Ed. Public Health Service, National Institutes of Health, Bethesda, Md. (1991)); and/or those residues from a “hypervariable loop” (e.g., residues 24-34 (L1), 50-56 (L2) and 89-97 (L3) in the V_{L}, and 26-32 (H1), 52-56 (H2) and 95-101 (H3) in the V_{H} when numbered in accordance with the Chothia numbering system; Chothia and Lesk, J. Mol. Biol. 196:901-917 (1987)); and/or those residues from a “hypervariable loop”/CDR (e.g., residues 27-38 (L1), 56-65 (L2) and 105-120 (L3) in the V_{L}, and 27-38 (H1), 56-65 (H2) and 105-120 (H3) in the V_{H} when numbered in accordance with the IMGT numbering system; Lefranc, M. P. et al. Nucl. Acids Res. 27:209-212 (1999), Ruiz, M. e al. Nucl. Acids Res. 28:219-221 (2000)). Optionally the antibody has symmetrical insertions at one or more of the following points 28, 36 (L1), 63, 74-75 (L2) and 123 (L3) in the VL, and 28, 36 (H1), 63, 74-75 (H2) and 123 (H3) in the VH when numbered in accordance with AHo; Honneger, A. and Plunkthun, A. J. Mol. Biol. 309:657-670 (2001)).

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies useful in the present invention may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

Monoclonal antibodies include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., Proc. Natl. Acad. Sci. USA, 81:6851-6855 (1984)). Also provided are variable domain antigen-binding sequences derived from human antibodies. Accordingly, chimeric antibodies of primary interest herein include antibodies having one or more human antigen binding sequences (e.g., CDRs) and containing one or more sequences derived from a non-human antibody, e.g., an FR or C region sequence. In addition, chimeric antibodies of primary interest herein include those comprising a human variable domain antigen binding sequence of one antibody class or subclass and another sequence, e.g., FR or C region sequence, derived from another antibody class or subclass. Chimeric antibodies of interest herein also include those containing variable domain antigen-binding sequences related to those described herein or derived from a different species, such as a non-human primate (e.g., Old World Monkey, Ape, etc). Chimeric antibodies also include primatized and humanized antibodies.

Furthermore, chimeric antibodies may comprise residues that are not found in the recipient antibody or in the donor antibody. These modifications are made to further refine antibody performance. For further details, see Jones et al., Nature 321:522-525 (1986); Riechmann et al., Nature 332:323-329 (1988); and Presta, Curr. Op. Struct. Biol. 2:593-596 (1992).

A “humanized antibody” is generally considered to be a human antibody that has one or more amino acid residues introduced into it from a source that is non-human. These non-human amino acid residues are often referred to as “import” residues, which are typically taken from an “import” variable domain. Humanization is traditionally performed following the method of Winter and co-workers (Jones et al., Nature, 321:522-525 (1986); Reichmann et al., Nature, 332:323-327 (1988); Verhoeyen et al., Science, 239:1534-1536 (1988)), by substituting import hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species.

A “human antibody” is an antibody containing only sequences present in an antibody naturally produced by a human. However, as used herein, human antibodies may comprise residues or modifications not found in a naturally occurring human antibody, including those modifications and variant sequences described herein. These are typically made to further refine or enhance antibody performance.

An “intact” antibody is one that comprises an antigen-binding site as well as a C_{L} and at least heavy chain constant domains, C_{H} 1, C_{H} 2 and C_{H} 3. The constant domains may be native sequence constant domains (e.g., human native sequence constant domains) or amino acid sequence variant thereof. Preferably, the intact antibody has one or more effector functions.

An “antibody fragment” comprises a portion of an intact antibody, preferably the antigen binding or variable region of the intact antibody. Examples of antibody fragments include Fab, Fab′, F(ab′)2, and Fv fragments; diabodies; linear antibodies (see U.S. Pat. No. 5,641,870; Zapata et al., Protein Eng. 8(10): 1057-1062 [1995]); single-chain antibody molecules; and multispecific antibodies formed from antibody fragments.

The phrase “functional fragment or analog” of an antibody is a compound having qualitative biological activity in common with a full-length antibody. For example, a functional fragment or analog of an anti-IgE antibody is one that can bind to an IgE immunoglobulin in such a manner so as to prevent or substantially reduce the ability of such molecule from having the ability to bind to the high affinity receptor, Fc_{ε}RI.

Papain digestion of antibodies produces two identical antigen-binding fragments, called “Fab′” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H} 1). Each Fab fragment is monovalent with respect to antigen binding, i.e., it has a single antigen-binding site. Pepsin treatment of an antibody yields a single large F(ab′)₂ fragment that roughly corresponds to two disulfide linked Fab fragments having divalent antigen-binding activity and is still capable of cross-linking antigen. Fab′ fragments differ from Fab fragments by having additional few residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments that have hinge cysteines between them. Other chemical couplings of antibody fragments are also known.

The “Fc” fragment comprises the carboxy-terminal portions of both H chains held together by disulfides. The effector functions of antibodies are determined by sequences in the Fc region, which region is also the part recognized by Fc receptors (FcR) found on certain types of cells.

“Fv” is the minimum antibody fragment that contains a complete antigen-recognition and -binding site. This fragment consists of a dimer of one heavy- and one light-chain variable region domain in tight, non-covalent association. From the folding of these two domains emanate six hypervariable loops (three loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv” also abbreviated as “sFv” or “scFv” are antibody fragments that comprise the V_{H} and V_{L} antibody domains connected into a single polypeptide chain. Preferably, the sFv polypeptide further comprises a polypeptide linker between the V_{H} and V_{L} domains that enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994); Borrebaeck 1995, infra.

The term “diabodies” refers to small antibody fragments prepared by constructing sFv fragments (see preceding paragraph) with short linkers (about 5-10 residues) between the V_{H} and V_{L} domains such that inter-chain but not intra-chain pairing of the V domains is achieved, resulting in a bivalent fragment, i.e., fragment having two antigen-binding sites. Bispecific diabodies are heterodimers of two “crossover” sFv fragments in which the V_{H} and V_{L} domains of the two antibodies are present on different polypeptide chains. Diabodies are described more fully in, for example, EP 404,097; WO 93/11161; and Hollinger et al., Proc. Natl. Acad. Sci. USA, 90:6444-6448 (1993).

As used herein, an antibody that “internalizes” is one that is taken up by (i.e., enters) the cell upon binding to an antigen on a mammalian cell (e.g., a cell surface polypeptide or receptor). The internalizing antibody will of course include antibody fragments, human or chimeric antibody, and antibody conjugates. For certain therapeutic applications, internalization in vivo is contemplated. The number of antibody molecules internalized will be sufficient or adequate to kill a cell or inhibit its growth, especially an infected cell. Depending on the potency of the antibody or antibody conjugate, in some instances, the uptake of a single antibody molecule into the cell is sufficient to kill the target cell to which the antibody binds. For example, certain toxins are highly potent in killing such that internalization of one molecule of the toxin conjugated to the antibody is sufficient to kill the infected cell.

As used herein, an antibody is said to be “immunospecific,” “specific for” or to “specifically bind” an antigen if it reacts at a detectable level with the antigen, preferably with an affinity constant, Kₐ, of greater than or equal to about 10⁴ M⁻¹, or greater than or equal to about 10⁵ M⁻¹, greater than or equal to about 10⁶ M⁻¹, greater than or equal to about 10⁷ M⁻¹, or greater than or equal to 10⁸ M⁻¹. Affinity of an antibody for its cognate antigen is also commonly expressed as a dissociation constant K_{D}, and in certain embodiments, HuM2e antibody specifically binds to M2e if it binds with a K_{D} of less than or equal to 10⁻⁴ M, less than or equal to about 10⁻⁵ M, less than or equal to about 10⁻⁶ M, less than or equal to 10⁻⁷ M, or less than or equal to 10⁻⁸ M. Affinities of antibodies can be readily determined using conventional techniques, for example, those described by Scatchard et al. (Ann. N.Y. Acad. Sci. USA 51:660 (1949)).

Binding properties of an antibody to antigens, cells or tissues thereof may generally be determined and assessed using immunodetection methods including, for example, immunofluorescence-based assays, such as immuno-histochemistry (IHC) and/or fluorescence-activated cell sorting (FACS).

An antibody having a “biological characteristic” of a designated antibody is one that possesses one or more of the biological characteristics of that antibody which distinguish it from other antibodies. For example, in certain embodiments, an antibody with a biological characteristic of a designated antibody will bind the same epitope as that bound by the designated antibody and/or have a common effector function as the designated antibody.

The term “antagonist antibody” is used in the broadest sense, and includes an antibody that partially or fully blocks, inhibits, or neutralizes a biological activity of an epitope, polypeptide, or cell that it specifically binds. Methods for identifying antagonist antibodies may comprise contacting a polypeptide or cell specifically bound by a candidate antagonist antibody with the candidate antagonist antibody and measuring a detectable change in one or more biological activities normally associated with the polypeptide or cell.

Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptor); and B cell activation.

By “agent” is meant any small molecule chemical compound, antibody, nucleic acid molecule, or polypeptide, or fragments thereof.

By “Collagen Triple Helix Repeat Containing 1” or “Cthrc1” is meant a polypeptide having at least about 85%, e.g., at least about 90%, at least about 95%, or at least about 99%, sequence identity to NCBI Accession No. NP_AAQ89273, or a fragment thereof that regulates metabolism. An exemplary sequence of human CTHRC1 is (SEQ ID NO: 1):

| | |
|---|---|
| 1 | mrpqgpaasp qrlrglllll llqlpapssa seipkgkqka qlrqrevvdl yngmclqgpa |
| 61 | gvpgrdgspg anvipgtpgi pgrdgfkgek geclresfee swtpnykqcs wsslnygidl |
| 121 | gkiaectftk mrsnsalrvl fsgslrlkcr naccqrwyft fngaecsgpl pieaiiyldq |
| 181 | gspemnstin ihrtssvegl cegigaglvd vaiwvgtcsd ypkgdastgw nsvsriiiee |
| 241 | lpk |

By a “nucleic acid encoding CTHRC1” is meant a nucleic acid having at least about 85%, e.g., at least about 90%, at least about 95%, or at least about 99%, sequence identity to NCBI Accession No. NM_138455 or NM_001256099. An exemplary nucleic acid encoding Cthrc1 is (SEQ ID NO: 2):

| | |
|---|---|
| 1 | gggagggaga gaggcgcgcg ggtgaaaggc gcattgatgc agcctgcggc ggcctcggag |
| 61 | cgcggcggag ccagacgctg accacgttcc tctcctcggt ctcctccgcc tccagctccg |
| 121 | cgctgcccgg cagccgggag ccatgcgacc ccagggcccc gccgcctccc cgcagcggct |
| 181 | ccgcggcctc ctgctgctcc tgctgctgca gctgcccgcg ccgtcgagcg cctctgagat |
| 241 | ccccaagggg aagcaaaagg cgcagctccg gcagagggag gtggtggacc tgtataatgg |
| 301 | aatgtgctta caagggccag caggagtgcc tggtcgagac gggagccctg gggccaatgg |
| 361 | cattccgggt acacctggga tcccaggtcg ggatggattc aaaggagaaa agggggaatg |
| 421 | tctgagggaa agctttgagg agtcctggac acccaactac aagcagtgtt catggagttc |
| 481 | attgaattat ggcatagatc ttgggaaaat tgcggagtgt acatttacaa agatgcgttc |
| 541 | aaatagtgct ctaagagttt tgttcagtgg ctcacttcgg ctaaaatgca gaaatgcatg |
| 601 | ctgtcagcgt tggtatttca cattcaatgg agctgaatgt tcaggacctc ttcccattga |
| 661 | agctataatt tatttggacc aaggaagccc tgaaatgaat tcaacaatta atattcatcg |
| 721 | cacttcttct gtggaaggac tttgtgaagg aattggtgct ggattagtgg atgttgctat |
| 781 | ctgggttggt acttgttcag attacccaaa aggagatgct tctactggat ggaattcagt |
| 841 | ttctcgcatc attattgaag aactaccaaa ataaatgctt taattttcat ttgctacctc |
| 901 | tttttttatt atgccttgga atggttcact taaatgacat tttaaataag tttatgtata |
| 961 | catctgaatg aaaagcaaag ctaaatatgt ttacagacca aagtgtgatt tcacactgtt |
| 1021 | tttaaatcta gcattattca ttttgcttca atcaaaagtg gtttcaatat tttttttagt |
| 1081 | tggttagaat actttcttca tagtcacatt ctctcaacct ataatttgga atattgttgt |
| 1141 | ggtcttttgt tttttctctt agtatagcat ttttaaaaaa atataaaagc taccaatctt |
| 1201 | tgtacaattt gtaaatgtta agaatttttt ttatatctgt taaataaaaa ttatttccaa |
| 1261 | caaccttaat atctttaaa |

Another exemplary nucleic acid encoding CTHRC1 is (SEQ ID NO: 3):

| | |
|---|---|
| 1 | agaaggttta aggccggaaa gggaaatgaa ggggcccggc gctaaccctc taaggacctg |
| 61 | ttttgcttct gtttaaacca aatgggcagt ctgtcattac acacaccctg ggtcttcata |
| 121 | tgtggccgcc aggtaggagc atcacagtca agctacggga gaaaacagtt tccaggaaac |
| 181 | tggaaatgaa cggcccgagt gctttccagg ggctcatctg tgggaagtat aatggaatgt |
| 241 | gcttacaagg gccagcagga gtgcctggtc gagacgggag ccctggggcc aatggcattc |
| 301 | cgggtacacc tgggatccca ggtcgggatg gattcaaagg agaaaagggg gaatgtctga |
| 361 | gggaaagctt tgaggagtcc tggacaccca actacaagca gtgttcatgg agttcattga |
| 421 | attatggcat agatcttggg aaaattgcgg agtgtacatt tacaaagatg cgttcaaata |
| 481 | gtgctctaag agttttgttc agtggctcac ttcggctaaa atgcagaaat gcatgctgtc |
| 541 | agcgttggta tttcacattc aatggagctg aatgttcagg acctcttccc attgaagcta |
| 601 | taatttattt ggaccaagga agccctgaaa tgaattcaac aattaatatt catcgcactt |
| 661 | cttctgtgga aggactttgt gaaggaattg gtgctggatt agtggatgtt gctatctggg |
| 721 | ttggtacttg ttcagattac ccaaaaggag atgcttctac tggatggaat tcagtttctc |
| 781 | gcatcattat tgaagaacta ccaaaataaa tgctttaatt ttcatttgct acctcttttt |
| 841 | ttattatgcc ttggaatggt tcacttaaat gacattttaa ataagtttat gtatacatct |
| 901 | gaatgaaaag caaagctaaa tatgtttaca gaccaaagtg tgatttcaca ctgtttttaa |
| 961 | atctagcatt attcattttg cttcaatcaa aagtggtttc aatatttttt ttagttggtt |
| 1021 | agaatacttt cttcatagtc acattctctc aacctataat ttggaatatt gttgtggtct |
| 1081 | tttgtttttt ctcttagtat agcattttta aaaaaatata aaagctacca atctttgtac |
| 1141 | aatttgtaaa tgttaagaat tttttttata tctgttaaat aaaaattatt tccaacaacc |
| 1201 | ttaatatctt taaa |

By “control” or “reference” is meant a standard of comparison. As used herein, “changed as compared to a control” sample or subject is understood as having a level of the analyte or diagnostic or therapeutic indicator to be detected at a level that is statistically different than a sample from a normal, untreated, or control sample. Control samples include, for example, cells in culture, one or more laboratory test animals, or one or more human subjects. Methods to select and test control samples are within the ability of those in the art. An analyte can be a naturally occurring substance that is characteristically expressed or produced by the cell or organism (e.g., an antibody, a protein) or a substance produced by a reporter construct (e.g, β-galactosidase or luciferase). Depending on the method used for detection, the amount and measurement of the change can vary. Determination of statistical significance is within the ability of those skilled in the art, e.g., the number of standard deviations from the mean that constitute a positive result.

As used herein, “detecting” and “detection” are understood that an assay performed for identification of a specific analyte in a sample, e.g., an antigen in a sample or the level of an antigen in a sample. The amount of analyte or activity detected in the sample can be none or below the level of detection of the assay or method.

By “diagnosing” as used herein refers to a clinical or other assessment of the condition of a subject based on observation, testing, or circumstances for identifying a subject having a disease, disorder, or condition based on the presence of at least one indicator, such as a sign or symptom of the disease, disorder, or condition. Typically, diagnosing using the method of the invention includes the observation of the subject for multiple indicators of the disease, disorder, or condition in conjunction with the methods provided herein. A diagnostic method provides an indicator that a disease is or is not present. A single diagnostic test typically does not provide a definitive conclusion regarding the disease state of the subject being tested.

By “effective amount” is meant the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The term “polynucleotide” or “nucleic acid” as used herein designates mRNA, RNA, cRNA, cDNA or DNA. As used herein, a “nucleic acid encoding a polypeptide” is understood as any possible nucleic acid that upon (transcription and) translation would result in a polypeptide of the desired sequence. The degeneracy of the nucleic acid code is well understood. Further, it is well known that various organisms have preferred codon usage, etc. Determination of a nucleic acid sequence to encode any polypeptide is well within the ability of those of skill in the art.

In some cases, a compound (e.g., small molecule) or macromolecule (e.g., nucleic acid, polypeptide, or protein) of the invention is purified and/or isolated. As used herein, an “isolated” or “purified” small molecule, nucleic acid molecule, polynucleotide, polypeptide, or protein (e.g., antibody or fragment thereof), is substantially free of other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (ribonucleic acid (RNA) or deoxyribonucleic acid (DNA), e.g., synthetic cDNA) is free of the genes or sequences that flank it in its naturally occurring state. Purified also defines a degree of sterility that is safe for administration to a human subject, e.g., lacking infectious or toxic agents.

Thus, an “isolated” or “purified” polypeptide can be in a cell-free solution or placed in a different cellular environment (e.g., expressed in a heterologous cell type). The term “purified” does not imply that the polypeptide is the only polypeptide present, but that it is essentially free (about 90-95%, up to 99-100% pure) of cellular or organismal material naturally associated with it, and thus is distinguished from naturally occurring polypeptide. Similarly, an isolated nucleic acid is removed from its normal physiological environment. “Isolated” when used in reference to a cell means the cell is in culture (i.e., not in an animal), either cell culture or organ culture, of a primary cell or cell line. Cells can be isolated from a normal animal, a transgenic animal, an animal having spontaneously occurring genetic changes, and/or an animal having a genetic and/or induced disease or condition. An isolated virus or viral vector is a virus that is removed from the cells, typically in culture, in which the virus was produced.

By “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally-occurring organic molecules with they are naturally associated.

By “isolated nucleic acid” is meant a nucleic acid that is free of the genes which flank it in the naturally-occurring genome of the organism from which the nucleic acid is derived. The term covers, for example: (a) a DNA which is part of a naturally occurring genomic DNA molecule, but is not flanked by both of the nucleic acid sequences that flank that part of the molecule in the genome of the organism in which it naturally occurs; (b) a nucleic acid incorporated into a vector or into the genomic DNA of a prokaryote or eukaryote in a manner, such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. Isolated nucleic acid molecules according to the present invention further include molecules produced synthetically, as well as any nucleic acids that have been altered chemically and/or that have modified backbones. For example, the isolated nucleic acid is a purified cDNA or RNA polynucleotide. Isolated nucleic acid molecules also include messenger ribonucleic acid (mRNA) molecules.

As used herein, “kits” are understood to contain at least one non-standard laboratory reagent for use in the methods of the invention in appropriate packaging, optionally containing instructions for use. The kit can further include any other components required to practice the method of the invention, as dry powders, concentrated solutions, or ready to use solutions. In some embodiments, the kit comprises one or more containers that contain reagents for use in the methods of the invention; such containers can be boxes, ampules, bottles, vials, tubes, bags, pouches, blister-packs, or other suitable container forms known in the art. Such containers can be made of plastic, glass, laminated paper, metal foil, or other materials suitable for holding reagents.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 .mu.g/ml denatured salmon sperm DNA (ssDNA). In a most preferred embodiment, hybridization will occur at 42° C. C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42.degree. C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

“Obtaining” is understood herein as manufacturing, purchasing, or otherwise coming into possession of.

As used herein, “operably linked” is understood as joined, preferably by a covalent linkage, e.g., joining an amino-terminus of one peptide, e.g., expressing an enzyme, to a carboxy terminus of another peptide, e.g., expressing a signal sequence to target the protein to a specific cellular compartment; joining a promoter sequence with a protein coding sequence, in a manner that the two or more components that are operably linked either retain their original activity, or gain an activity upon joining such that the activity of the operably linked portions can be assayed and have detectable activity, e.g., enzymatic activity, protein expression activity.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, a-tocopherol, and the like; and metal chelating agents, such as citric acid, ethylenediamine tetraacetic acid (EDTA), sorbitol, tartaric acid, phosphoric acid, and the like. Formulations of the present invention include those suitable for oral, nasal, topical, transdermal, buccal, sublingual, intramuscular, intracardiac, intraperotineal, intrathecal, intracranial, rectal, vaginal and/or parenteral administration. The formulations may conveniently be presented in unit dosage form and may be prepared by any methods well known in the art of pharmacy. The amount of active ingredient that can be combined with a carrier material to produce a single dosage form will generally be that amount of the compound that produces a therapeutic effect.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

As used herein, “plurality” is understood to mean more than one. For example, a plurality refers to at least two, three, four, five, or more.

A “polypeptide” or “peptide” as used herein is understood as two or more independently selected natural or non-natural amino acids joined by a covalent bond (e.g., a peptide bond). A peptide can include 2, 3, 4, 5, 6, 7, 8, 9, 10, 11, 12, 13, 14, 15, 16, 17, 18, 19, 20, or more natural or non-natural amino acids joined by peptide bonds. Polypeptides as described herein include full length proteins (e.g., fully processed proteins) as well as shorter amino acids sequences (e.g., fragments of naturally occurring proteins or synthetic polypeptide fragments). Optionally the peptide further includes one or more modifications such as modified peptide bonds, i.e., peptide isosteres, and may contain amino acids other than the 20 gene-encoded amino acids. The polypeptides may be modified by either natural processes, such as posttranslational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formulation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, Proteins, Structure and Molecular Properties, 2nd ed., T. E. Creighton, W.H. Freeman and Company, New York (1993); Posttranslational Covalent Modification of Proteins, B. C. Johnson, ed., Academic Press, New York, pgs. 1-12 (1983); Seifter et al., Meth. Enzymol 182:626-646 (1990); Rattan et al., Ann. N.Y. Acad. Sci. 663:48-62 (1992)).

The term “reduce” or “increase” is meant to alter negatively or positively, respectively, by at least 5%. An alteration may be by 5%, 10%, 25%, 30%, 50%, 75%, or even by 100%.

A “sample” as used herein refers to a biological material that is isolated from its environment (e.g., blood or tissue from an animal, cells, or conditioned media from tissue culture) and is suspected of containing, or known to contain an analyte, such as a protein. A sample can also be a partially purified fraction of a tissue or bodily fluid. A reference sample can be a “normal” sample, from a donor not having the disease or condition fluid, or from a normal tissue in a subject having the disease or condition. A reference sample can also be from an untreated donor or cell culture not treated with an active agent (e.g., no treatment or administration of vehicle only). A reference sample can also be taken at a “zero time point” prior to contacting the cell or subject with the agent or therapeutic intervention to be tested or at the start of a prospective study.

A “subject” as used herein refers to an organism. In certain embodiments, the organism is an animal. In certain embodiments, the subject is a living organism. In certain embodiments, the subject is a cadaver organism. In certain preferred embodiments, the subject is a mammal, including, but not limited to, a human or non-human mammal. In certain embodiments, the subject is a domesticated mammal or a primate including a non-human primate. Examples of subjects include humans, monkeys, dogs, cats, mice, rats, cows, horses, goats, and sheep. A human subject may also be referred to as a patient.

A “subject sample” can be a sample obtained from any subject, typically a blood or serum sample, however the method contemplates the use of any body fluid or tissue from a subject. The sample may be obtained, for example, for diagnosis of a specific individual for the presence or absence of a particular disease or condition.

A subject “suffering from or suspected of suffering from” a specific disease, condition, or syndrome has a sufficient number of risk factors or presents with a sufficient number or combination of signs or symptoms of the disease, condition, or syndrome such that a competent individual would diagnose or suspect that the subject was suffering from the disease, condition, or syndrome. Methods for identification of subjects suffering from or suspected of suffering from conditions associated with diminished cardiac function is within the ability of those in the art. Subjects suffering from, and suspected of suffering from, a specific disease, condition, or syndrome are not necessarily two distinct groups.

As used herein, “susceptible to” or “prone to” or “predisposed to” a specific disease or condition and the like refers to an individual who based on genetic, environmental, health, and/or other risk factors is more likely to develop a disease or condition than the general population. An increase in likelihood of developing a disease may be an increase of about 10%, 20%, 50%, 100%, 150%, 200%, or more.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Ranges provided herein are understood to be shorthand for all of the values within the range. This includes all individual sequences when a range of SEQ ID NOs: is provided. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Unless specifically stated or obvious from context, as used herein, the term “or” is understood to be inclusive.

Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Unless specifically stated or obvious from context, as used herein, the term “or” is understood to be inclusive. Unless specifically stated or obvious from context, as used herein, the terms “a”, “an”, and “the” are understood to be singular or plural.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All published foreign patents and patent applications cited herein are incorporated herein by reference. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

FIG. 1A-FIG. 1J is a series of photomicrographs and immunoblots showing that CTHRC1 is expressed in bone by osteoblasts and osteocytes, not osteoclasts. FIG. 1A shows that monoclonal anti-CTHRC1 antibody reveals CTHRC1 in osteoblasts (arrowheads) and osteocytes of trabecular and cortical bone in wildtype mice, whereas multinucleated osteoclasts (arrows) do not express CTHRC1. FIG. 1B shows that confocal imaging of TRAP (red) and CTHRC1 (green) immunohistochemistry of a bone trabeculum shows no overlap, demonstrating that CTHRC1 is not expressed in cells expressing the osteoclast marker TRAP (nuclear stain with DAPI). FIG. 1C shows the distinct localization of CTHRC1 in canaliculi of some osteocytes. FIG. 1D and FIG. 1E show that CTHRC1 is found in the thin cytoplasmic processes of osteocytes (arrow) Immunoreactive CTHRC1 appears to be present in the wider canaliculi (arrows) indicating secretion of CTHRC1 into the interstitial fluid. FIG. 1F shows that prominent accumulation of CTHRC1 is frequently seen around venules (arrows). FIG. 1G shows that CTHRC1 is not detectable in trabecular bone of Cthrc1 knockout mice demonstrating antibody specificity. FIG. 1H shows that Cthrc1 expression was analyzed by RT-PCR using mRNA isolated from bone fractions of metaphysis and proximal epiphysis (trabecular bone), diaphysis (cortical bone), as well as bone marrow, which does not express Cthrc1. FIG. 1I shows that Cthrc1 mRNA expression was analyzed in primary bone marrow-derived mesenchymal stromal cells during osteogenic differentiation. FIG. 1J shows that western blot analysis of CTHRC1 from femur and marrow lysates obtained from wildtype and Cthrc1 null mice shows CTHRC1 in bone but not the marrow fraction.

FIG. 2A-FIG. 2B is a series of photomicrographs showing that bone formation and bone strength are reduced in Cthrc1 null mice. FIG. 2A is a von Kossa staining of proximal tibia sections of Cthrc1 null and wildtype mice is shown. FIG. 2B is a representative micro-CT images of cortical and trabecular bone show decreased trabecular bone in Cthrc1 knockout mice.

FIG. 3A-FIG. 3M is a series of photographs and bar charts showing that CTHRC1 does not affect osteogenic differentiation of bone marrow derived stromal cells or bone formation in vitro. Alizarin red staining shows similar mineral content in bone marrow-derived stromal cells isolated from (FIG. 3A) wildtype or (FIG. 3B) Cthrc1 null mice after induction of osteogenic differentiation. FIG. 3C shows that bone marrow stromal cell cultures from wildtype and Cthrc1^{-/-} mice form similar amounts of bone in vitro as determined by von Kossa staining. Alizarin red staining (FIG. 3D and FIG. 3E) and alkaline phosphatase activity staining (FIG. 3F and FIG. 3G) show that differentiation of bone marrow-derived stromal cells isolated from Cthrc1^{-/-} mice in the presence of control conditioned-medium (Control-CM) or hCTHRC1 (hCTHRC1-CM) is similar. von Kossa staining showed similar results (not shown). FIG. 3H shows that bone marrow stromal cell proliferation was unaffected by the presence or absence of CTHRC1 with similar numbers of colonies formed. FIG. 3I and FIG. 3J show that following transduction with control adenovirus (b-gal) or hCTHRC1 adenovirus bone marrow-derived stromal cells isolated from Cthrc1 null mice revealed similar osteogenic differentiation potential. FIG. 3K and FIG. 3L show that calvarial osteoblast cell cultures from Cthrc1 null mice showed reduced bone formation as determined by decreased Alizarin red and von Kossa staining. FIG. 3M shows that CTHRC1 was secreted into the medium by wildtype osteoblast cultures as determined by ELISA (GM5-day 5 in growth medium, DM3-day 3 in differentiation medium). Differentiation was performed over a 20-day period for all experiments. *p<0.05, ***p<0.005.

FIG. 4A-FIG. 4I show that Cthrc1 negatively regulates osteoclastogenesis in vivo and in vitro. FIG. 4A-FIG. 4C show that histochemistry for TRAP was performed on differentiated osteoclasts of bone marrow-derived monocytes derived from Cthrc1 null mice following 9 days of differentiation in the presence of control conditioned medium (CM, Control-CM) or hCTHRC1-containing CM. FIG. 4C shows that TRAP positive multinucleated (>3 nuclei) osteoclasts were counted in each well. Data represent means±SD and are averages of ≥3 experiments. FIG. 4D, FIG. 4E, and FIG. 4F show that in vitro bone resorption assays performed with osteoclast cultures from wildtype mice showed reduced bone resorption (pits in grey) in the presence of CTHRC1 (FIG. 4E) compared to control treated cells (FIG. 4D) with quantification shown in (FIG. 4F). Unresorbed bone matrix appears black. FIG. 4G and FIG. 4H show that osteoclasts derived from bone marrow monocytes isolated from wildtype and Cthrc1 knockout mice differentiated similarly as determined by TRAP histochemistry performed after 7 days of differentiation. FIG. 4I shows the number of TRAP positive multinucleated osteoclasts (>3 nuclei) were similar for both genotypes. Data represent means±SEM. ***p<0.005, ****p<0.001, N.S.=not significant.

FIG. 5A-FIG. 5L is a series of bar charts that show that Cthrc1 negatively regulates osteoclastogenic marker gene expression in vivo and in vitro. FIG. 5A-5F shows bone marrow-derived monocytes from Cthrc1 knockout mice were cultured in osteoclastogenic differentiation medium containing control-CM (CON-CM) or hCTHRC1-CM for the indicated time periods. mRNA expression of Trap (FIG. 5A), c-Fos (FIG. 5B), Rank (FIG. 5C), Opg (FIG. 5D), CtsK (FIG. 5E), and Pparγ (FIG. 5F) was analyzed by real-time RT-PCR. FIG. 5G-5L shows mRNA expression of Trap (FIG. 5H), c-Fos (FIG. 5I), Rankl (FIG. 5G), Opg (FIG. 5K), Nfatc1 (FIG. 5J), and Rank (FIG. 5L) in femurs of 3 month old wildtype and Cthrc1 knockout mice was analyzed by real-time RT-PCR. Data represent means±SEM with ** indicating p<0.01 and *** indicating p<0.005.

FIG. 6A-FIG. 6M is a series of photomicrographs, bar graphs, and immunoblots that show the effects of CTHRC1 on RANKL-induced osteoclastogenic differentiation and intracellular signaling transduction in RAW264.7 cells. FIG. 6A-FIG. 6C show RAW264.7 cells were differentiated with RANKL for 5 days and the time-dependent presence of hCTHRC1 to inhibit osteoclast differentiation was assessed. TRAP-positive multinucleated cells were quantified. Data represent means±SEM of ≥3 experiments with hCTHRC1-CM versus control-CM treated cells. FIG. 6D shows that osteoclast differentiation of RAW264.7 cells in the presence of hCTHRC1 (hCTHRC1-CM) is inhibited. Depleting hCTHRC1-CM with anti-CTHRC1 monoclonal antibodies abolishes this effect, demonstrating specificity. FIG. 6E shows the effectiveness of hCTHRC1-CM depletion was verified by measuring hCTHRC1 levels before and after depletion using an established ELISA. FIG. 6F shows that RAW264.7 cells were stimulated with RANKL for the indicated length of time in the presence of hCTHRC1 or control medium. Western blotting of cell lysates shows reduced activation of NFκB (p-NFκB), reduced degradation of IκBα and reduced phosphorylation of IκB□ (p-IκBα□ in the presence of hCTHRC1 (asterisks). In FIG. 6G-FIG. 6H quantification of immunoblot data marked with * in FIG. 6F are shown. FIG. 6J shows that hCTHRC1 inhibits RANKL-induced NFκB-dependent luciferase reporter activity. FIG. 6K shows a western blot analysis of IκBα levels in femur lysates from wildtype and Cthrc1 null mice shows significantly reduced levels in the mutants. FIG. 6L and FIG. 6M show that RAW264.7 cells treated with control-CM or hCTHRC1-CM were transfected with control vector (pcDNA3) or a vector expressing a constitutively active form of IKKβ (ca-IKKβ), which reversed inhibition of osteoclast differentiation by hCTHRC1. All data represent means±SEM of replicates ≥3 with *=p<0.05, **=p<0.01, and ***=p<0.005.

FIG. 7A is an immunoblot analysis of a time course of ERK1/2 activation (p-ERK1/2) in RAW264.7 cells stimulated with RANKL in the presence of control-CM or hCTHRC1-CM is shown. The corresponding quantification of the signal marked with * is shown in (FIG. 7C). In FIG. 7B p-ERK1/2 levels in RAW264.7 cells growing in differentiation medium (DM) for up to 2 days are shown with corresponding quantification shown in (FIG. 7D). FIG. 7E shows that hCTHRC1 inhibits RANKL-induced AP-1 luciferase reporter activity in RAW264.7 cells transfected with the AP-1 promoter reporter plasmid. FIG. 7F is a western blot analysis of p-ERK1/2 level in femur lysates from wildtype and Cthrc1 null mice. All data represent means±SEM of replicates ≥3 with *=p<0.05.

FIG. 8A-FIG. 8H is a seroes photomicrographs and dot plots showing that the loss of Cthrc1 exacerbates arthritis in a collagen antibody-induced arthritis model. FIG. 8A shows that CTHRC1 immunohistochemistry was performed on sections of knee joints from wildtype (WT) and (FIG. 8B) Cthrc1 null mice 21 days after collagen antibody injection. Fibroblast-like activated synoviocytes of the pannus show prominent expression of CTHRC1, most pronounced in cells near the eroded bone surface. Absence of immunoreactive CTHRC1 in Cthrc1 null bones (FIG. 8B) demonstrates specificity of the staining. Representative images show hematoxylin-eosin stained sections of knee (FIG. 8C and FIG. 8D) and ankle joints (FIG. 8E and FIG. 8F) from Cthrc1 null and wildtype mice with extensive inflammatory cell infiltrates, large pannus formation and bone erosion in FIG. 8D and FIG. 8F. FIG. 8G shows the degree of inflammation in knee joints and ankle joints was scored in a blinded fashion on a scale from 0 to 4 (“no”-“max” inflammation). Cthrc1 null mice showed significantly larger pannus when compared to wildtype mice (Mann-Whitney U test). Each symbol represents a separately scored histological section at 150× magnification. Horizontal lines are medians with interquartile range.

FIG. 9 is an immunoblot showing the effects of hCTHRC1 on M-CSF-induced early intracellular signaling transduction in bone marrow derived monocytes. Bone marrow derived monocytes were harvested and cultured as described in Materials and Methods. Immnoblotting was performed on cell lysates stimulated with M-CSF for the indicated lengths of time in the presence of hCthrc1-CM or control-CM. M-CSF signaling via JNK, p38 or ERK was unaffected by hCTHRC1.

FIG. 10 is an immunoblot showing the effects CTHRC1 on RANKL-induced early intracellular signaling transduction in bone marrow derived monocytes. Bone marrow derived monocytes were harvested and cultured as described in Materials and Methods. Immnoblotting was performed on cell lysates stimulated with RANKL for the indicated lengths of time in the presence of hCthrc1-CM or control-CM. RANKL signaling via JNK or p38 was unaffected by hCTHRC1.

### DETAILED DESCRIPTION

The invention is based, at least in part, on the surprising discovery that CTHRC1 inhibits inflammation and osteoclast function. Specifically, CTHRC1 inhibits osteoclast differentiation and collagen antibody-induced arthritis.

Collagen triple helix repeat containing 1 (Cthrc1) was originally discovered in a screen for sequences induced in injured arteries, where it was identified that Cthrc1 expressed in adventitial cells of remodeling arteries but not in uninjured vessels (1). Subsequent studies also demonstrated that CTHRC1 is characteristically expressed by the activated fibroblast associated with wound healing as well as cancer-activated fibroblast (2,3). Kimura et al. (4) first reported that Cthrc1 null mice exhibit reduced bone mass, and in vitro osteogenic differentiation of bone marrow stromal cells revealed that endogenously expressed Cthrc1 is required for effective osteogenic differentiation by affecting cell proliferation and differentiation (4). In contrast, it was recently reported that CTHRC1 stimulates bone formation in vitro and that it functions as a coupling factor in vivo, produced by mature actively resorbing osteoclasts. The key difference between the two studies is the identity of the CTHRC1-producing cell, which in the absence of specific and reliable antibody reagents has remained controversial.

To understand the in vivo function of CTHRC1 during adulthood, a targeted Cthrc1 null allele was recently generated by replacing exon 2, 3 and 4 with a neomycin cassette (3). It was reported that on the C57BL/6J background, Cthrc1 null mice develop fatty livers with extensive macrovesicular steatosis (5). Using highly specific monoclonal antibodies it was identified that CTHRC1 is prominently expressed by neuroendocrine cells of the hypothalamus (5), which are likely sources contributing to circulating levels of CTHRC1 detectable in plasma (2). Bone was also identified as another tissue that constitutively expresses CTHRC1 in the adult (3). The recent study by Takeshita et al (6) reported that deletion of floxed Cthrc1 alleles with a cathepsin-K-Cre strain (Ctsk) could fully recapitulate the bone phenotype observed in global knockout mice, whereas an osteoblast lineage specific knockout of Cthrc1 did not recapitulate the bone phenotype. A recent study, however, demonstrated that Ctsk is also expressed in mesenchymal progenitor cells, indicating Ctsk-Cre mediated Cthrc1 deletion will not be restricted to osteoclasts (7).

To eliminate potential effects of circulating CTHRC1 on bone formation the present study investigated the functions of CTHRC1 in bone using a global Cthrc1 null mutant mouse on a pure C57BL/6J background. In addition, analyses were performed on both males and females. As described in detail herein, using primary cultures of bone marrow stromal cells and calvarial osteoblasts for osteogenic differentiation, and bone marrow-derived monocytes together with RAW264.7 cells for osteoclastogenic differentiation the mechanism of CTHRC1 function in bone homeostasis was elucidated.

Collagen triple helix repeat-containing 1 (Cthrc1) has previously been implicated in osteogenic differentiation and positive regulation of bone mass, however, the underlying mechanisms remain unclear. Here, the bone phenotype of a Cthrc1 null mouse strain was characterized using bone histomorphometry, μCT analysis and functional readouts for bone strength. In male Cthrc1 null mice both trabecular bone as well as cortical bone formation was impaired, whereas in female Cthrc1 null mice only trabecular bone parameters were altered. Highly specific monoclonal antibodies revealed that CTHRC1 is expressed by osteocytes and osteoblasts, but not osteoclasts. Furthermore, Cthrc1 null mice exhibited increased bone resorption with increased number of osteoclast and increased osteoclast activity together with enhanced expression of osteoclastogenic genes such as c-Fos, Rankl, Trap, and Nfatc1. Differentiation of bone marrow-derived monocytes isolated from Cthrc1 null mice differentiated into osteoclasts as effectively as those from wildtype mice. In the presence of CTHRC1 osteoclastogenic differentiation of bone marrow-derived monocytes was dramatically inhibited as was functional bone resorption by osteoclasts. This process was accompanied by downregulation of osteoclastogenic marker genes, indicating that extrinsically derived CTHRC1 is required for such activity. In vitro, CTHRC1 had no effect on osteogenic differentiation of bone marrow stromal cells, however, calvarial osteoblasts from Cthrc1 null mice exhibited reduced osteogenic differentiation compared to osteoblasts from wildtypes. In a collagen antibody-induced arthritis model Cthrc1 null mice suffered significantly more severe inflammation and joint destruction than wildtypes, suggesting that CTHRC1 expressed by the activated synoviocytes has anti-inflammatory effects. Mechanistically, it was found that CTHRC1 inhibited NFκB activation by preventing IκBα degradation while also inhibiting ERK1/2 activation. Collectively, these studies demonstrate that CTHRC1 secreted from osteocytes and osteoblasts functions as an inhibitor of osteoclast differentiation via inhibition of NFκB-dependent signaling. Furthermore, the data suggest that CTHRC1 has potent anti-inflammatory properties that limit arthritic joint destruction.

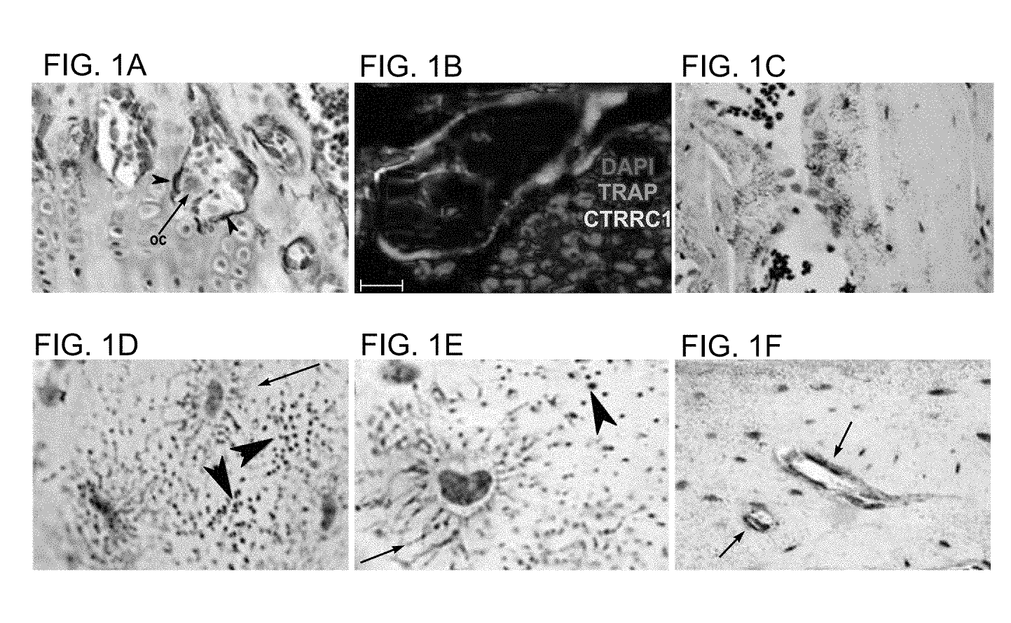

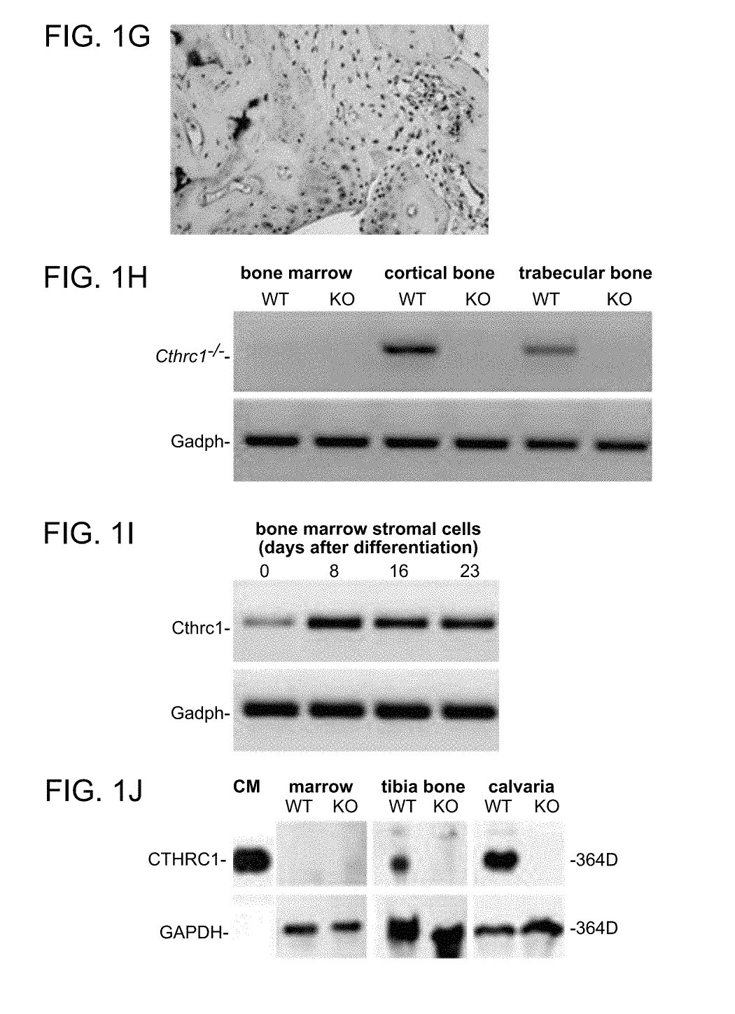

In the present study, it was established that CTHRC1 is a key factor secreted by osteoblasts and some osteocytes (FIG. 1). It was also demonstrated that CTHRC1 functions as a potent negative regulator of osteoclastogenesis by inhibiting RANKL-stimulated NFκB signaling activation and ERK1/2 phosphorylation. Bone formation in vivo was reduced in Cthrc1 null mice. Although bone marrow stromal cells from Cthrc1 null mutants can differentiate into osteoblasts as efficiently as wildtype cells in vitro, primary cultures of calvarial osteoblasts from newborn Cthrc1 null mice differentiated less efficiently than their wildtype controls. These results indicate that cell-autonomous effects of CTHRC1 with regards to bone formation are cell type dependent.

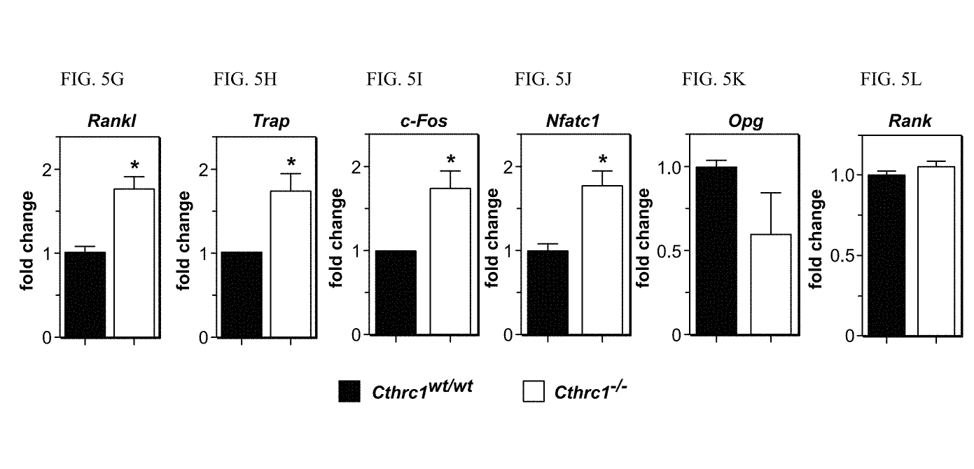

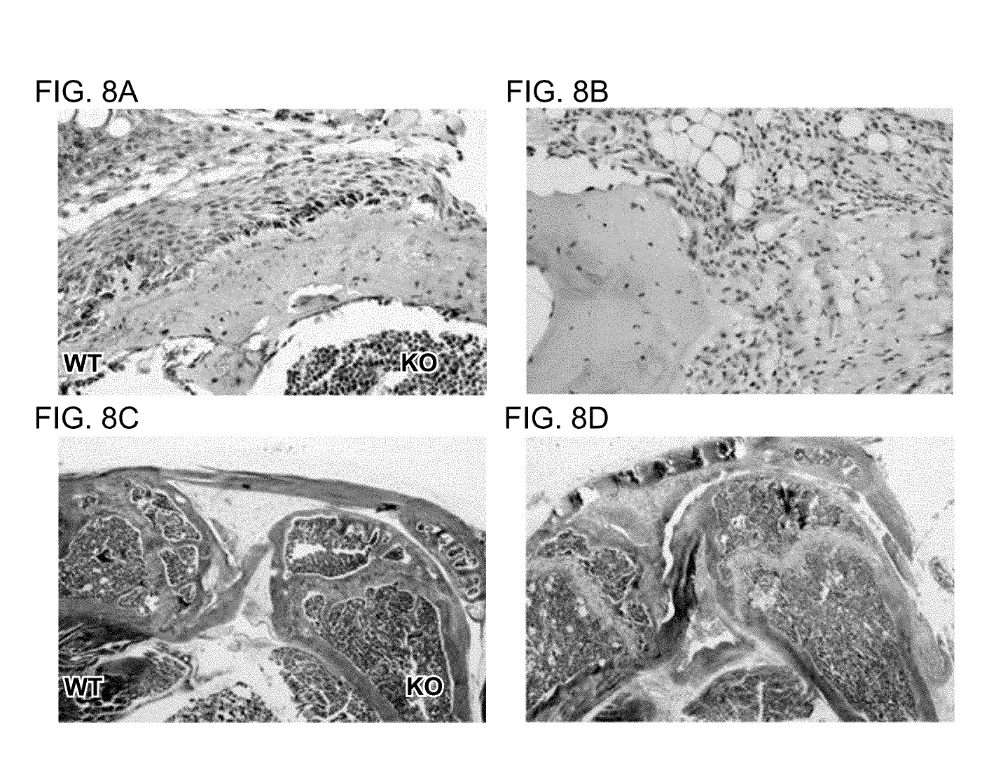

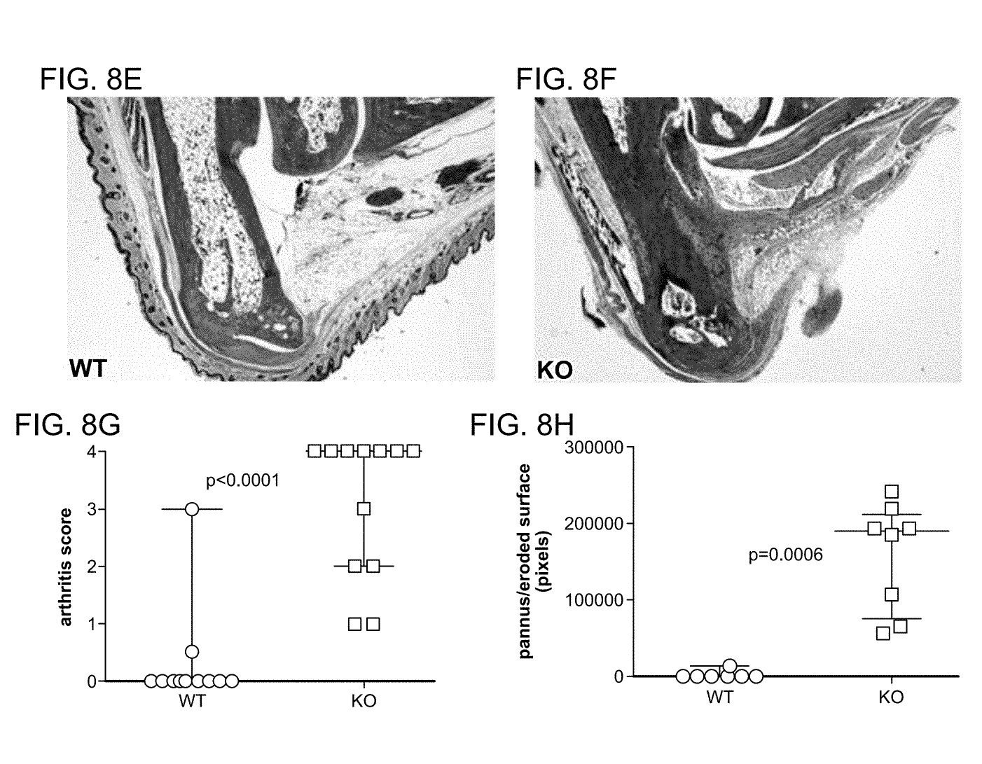

As described in detail below, control of bone resorption is mediated by RANKL, which was expressed at higher levels in bones from Cthrc1 null mice (FIG. 5B), suggesting that CTHRC1 may also affect bone mass by suppressing Rankl expression in addition to RANK signaling. A concomitant reduction in expression of the RANKL decoy receptor Opg (FIG. 5B) could further increase RANK signaling in Cthrc1 null mice promoting bone resorption. Whether RANKL expression is altered also in other tissues remains to be determined. RANKL plays additional important roles in the maturation and activation of the immune system (17,18) with implications for therapies targeting rheumatoid arthritis. In the collagen II antibody-induced arthritis model, severely exacerbated arthritis was observed in Cthrc1 null mice with extensive inflammatory cell infiltration and pannus formation with extensive bone destruction. Only one of the wildtype mice showed arthritis in some joints by histology whereas all Cthrc1 null mice developed arthritis, the majority with a maximal arthritis score (FIG. 8). These findings indicate that CTHRC1 has potent anti-inflammatory functions, suggestive of a broader role in immune responses.

As described in detail below, bones from males are more severely affect by the loss of Cthrc1 than bones from females. This is supported not only by measurements of bone parameters but also by functional testing of bone strength (Table 1 and 2). Studies addressing the underlying mechanism for this gender discrepancy are beyond the scope of this study.

TABLE 1
Histomorphometry was performed on tibiae of 8 week old Cthrc1 null mice
(Cthrcl^{tm1Vli}) and corresponding wildtype mice (WT) on the C57BL/6J background.

| | Males | | Females | |
|---|---|---|---|---|
| | WT | Cthrc1^{tm1Vli} | WT | Cthrc1^{tm1Vli} |
| | (n = 6) | (n = 6) | (n = 6) | (n = 6) |
| BV/TV (%) | 13.0 ± 3.91 | 8.60 ± 1.72* | 10.3 ± 3.94 | 5.60 ± 0.91* |
| Tb.Th (μm) | 33.3 ± 5.33 | 24.9 ± 3.03** | 33.6 ± 5.52 | 27.3 ± 3.33* |
| Tb.N (/mm) | 3.85 ± 0.58 | 3.45 ± 0.43 | 3.00 ± 0.71 | 2.06 ± 0.32* |
| Tb.Sp (μm) | 232 ± 43.6 | 269 ± 36.7 | 317 ± 89.9 | 470 ± 79.2* |
| MAR (μm/day) | 1.91 ± 0.30 | 1.39 ± 0.30* | 1.98 ± 0.24 | 1.32 ± 0.12** |
| MS/BS (%) | 27.9 ± 5.20 | 27.6 ± 1.70 | 25.6 ± 3.10 | 21.8 ± 3.60 |
| BFR/BV (%/year) | 1157 ± 154.6 | 1088 ± 215.6 | 1155 ± 137.0 | 761.4 ± 193.3** |
| BFR/BS (μm³/μm²/year) | 193 ± 38.7 | 140 ± 30.3* | 185 ± 27. | 106 ± 24.9** |
| N.Ob/B.Pm (/mm) | 8.40 ± 1.68 | 5.82 ± 0.86** | 12.5 ± 3.83 | 9.40 ± 4.20 |
| Ob.S/B.Pm (%) | 13.3 ± 3.15 | 9.10 ± 1.69* | 20.9 ± 6.51 | 14.4 ± 6.23 |
| OS/BS (%) | 10.4 ± 3.96 | 3.57 ± 1.47** | 8.86 ± 2.68 | 4.45 ± 1.28** |
| O.Th (μm) | 6.49 ± 0.73 | 5.52 ± 0.73* | 6.49 ± 0.98 | 5.01 ± 0.97* |
| N.Oc/B.Pm (/mm) | 1.62 ± 0.22 | 2.28 ± 0.38** | 2.52 ± 0.56 | 3.42 ± 0.75* |
| Oc.S/B.Pm (%) | 4.52 ± 1.11 | 6.65 ± 1.32* | 7.39 ± 1.83 | 9.82 ± 2.47 |
| ES/BS (%) | 2.29 ± 0.68 | 3.82 ± 0.86** | 2.76 ± 0.87 | 5.04 ± 1.39** |

Data are means ± SD.
*denotes statistically significant with p < 0.05;
**denotes statistically significant with p < 0.01.

TABLE 2
Results for three-point bending and μCT analyses performed on
femurs from 8 week old Cthrc1 null mice (Cthrc1^{tm1Vli}) and corresponding
wildtype mice (WT) on the C57BL/6J background are shown.

| Males | | Females | |
|---|---|---|---|
| WT | Cthrc1^{tm1Vli} | WT | Cthrc1^{tm1Vli} |
| (n = 6) | (n = 6) | (n = 6) | (n = 6) |

Three-point bending data

| | | | | |
|---|---|---|---|---|
| Max. Moment (N-mm) | 30.15 ± 4.49 | 21.38 ± 3.40** | 23.29 ± 1.87 | 20.30 ± 3.12 |
| Bending stiffness (N-mm²) | 838 ± 142 | 566 ± 159** | 744 ± 131 | 641 ± 115 |
| Estimated Modulus (GPa) | 6.43 ± 1.25 | 6.19 ± 1.42 | 8.33 ± 1.58 | 7.71 ± 0.64 |

Mid-diaphysis μCT data

| | | | | |
|---|---|---|---|---|
| Femur length (mm) | 14.55 ± 0.20 | 13.75 ± 0.20** | 13.84 ± 0.61 | 13.62 ± 0.40 |
| Ct.Th (mm) | 0.161 ± 0.011 | 0.123 ± 0.006** | 0.132 ± 0.012 | 0.121 ± 0.013 |
| CtTMD (mgHA/cm³) | 1030 ± 8 | 1014 ± 6** | 1012 ± 18 | 1040 ± 14 |
| CtAr (mm²) | 0.774 ± 0.114 | 0.555 ± 0.037** | 0.574 ± 0.042 | 0.518 ± 0.061 |
| Ma.Ar (mm²) | 1.201 ± 0.125 | 1.208 ± 0.058 | 1.103 ± 0.069 | 1.125 ± 0.023 |
| Tt.Ar (mm²) | 1.975 ± 0.238 | 1.763 ± 0.088* | 1.677 ± 0.059 | 1.644 ± 0.066 |
| Ct.Ar/Tt.Ar (%) | 39.10 ± 1.29 | 31.48 ± 1.07** | 34.26 ± 2.70 | 31.47 ± 2.55 |
| Ct. Porosity (%) | 0.442 ± 0.166 | 0.613 ± 0.088* | 0.518 ± 0.138 | 0.500 ± 0.034 |
| pMOI (mm⁴) | 0.424 ± 0.111 | 0.279 ± 0.030** | 0.267 ± 0.020 | 0.239 ± 0.033 |
| Iₘₐₓ (mm⁴) | 0.289 ± 0.078 | 0.187 ± 0.021** | 0.177 ± 0.014 | 0.156 ± 0.020 |
| Iₘᵢₙ (mm⁴) | 0.135 ± 0.034 | 0.092 ± 0.010** | 0.090 ± 0.009 | 0.083 ± 0.013 |

Data are means ± SD.
*Denotes statistically significant with p < 0.05;
**denotes statistically significant with p < 0.01.

Previous studies reported that CTHRC1 is a positive regulator of osteoblastic bone formation in vivo and in vitro (4,6). Using primary cultured bone marrow stromal cells in vitro, Kimura et al. reported that Cthrc1 functions intrinsically as an autocrine protein to stimulate osteoblast proliferation and osteogenic differentiation (4). In contrast, Takeshita et al. reported that CTHRC1 acts as a coupling factor, expressed only by mature bone-resorbing osteoclasts, to stimulate osteoblastogenesis of calvarial osteoblastic cells in vitro (6). To reconcile these differing reports, panels of both rabbit and mouse monoclonal antibodies were generated and validated exhaustively for various applications, including immunohistochemistry on tissues from Cthrc1 null mice as negative controls. The data unequivocally demonstrate that CTHRC1 is not expressed by osteoclasts but rather by osteoblasts and some osteocytes (FIG. 1). The data also show that during in vitro culture of bone marrow stromal cells Cthrc1 gene expression gradually increases with progressive osteogenic differentiation (FIG. 1). This is consistent with earlier reports of CTHRC1 production in activated stromal cells of remodeling muscle, heart, vasculature, as well as tumor stroma (2). In addition, it was also recently demonstrated that progenitor cells derived from the stromal vascular fraction of adipose tissue express Cthrc1 (5). Considering the association of CTHRC1 expression with tissue remodeling, it is perhaps not surprising that bone as a constantly remodeling tissue is the only tissue constitutively expressing CTHRC1 along with brain.

While it was identified that CTHRC1 has cell type dependent effects on bone formation in vitro, an autocrine mechanism for stimulating bone formation in vivo may have limited impact considering that cortical bone formation is fairly normal in Cthrc1 null mice, especially in females (Table 1 and 2). The more pronounced phenotype in trabecular bone of Cthrc1 null mice is, however, consistent with a role of CTHRC1 in osteoclasts because per unit of bone volume more osteoclasts are present in trabecular bone than cortical bone.

Osteocyte cytoplasmic processes are on average 104 nm in diameter compared to the average 259 nm diameter of the canaliculi (19). Higher power views of CTHRC1 localization in bone reveal CTHRC1 in the thinner cytoplasmic processes close to the osteocyte cell body (FIG. 1D and E, arrows). The presence of CTHRC1 within the wider structure of the canaliculi (FIG. 1D and E, arrowheads) suggests that CTHRC1 is secreted from the osteocyte into the canalicular system, which is part of the interstitial space. The localization of CTHRC1 in the osteocyte canaliculi along with often prominent accumulations of CTHRC1 around intraosseal venules (FIG. 1F, arrows) would support the concept that osteocyte-derived CTHRC1 is contributing to circulating CTHRC1 levels observed in humans (2). The functions of circulating CTHRC1 are currently unknown, however, the association of elevated CTHRC1 levels in subjects known to have variant alleles of the melanocortin receptor MC1R suggests that the melanocortin system may be involved (2). CTHRC1 plasma levels in wildtype mice are typically below the detection limit of the assay.

Takeshita et al. previously reported that global deletion of Cthrc1 as well as osteoclast-specific deletion of Cthrc1 both lead to lower bone mass due to decreased bone formation, while osteoblast-specific deletion of Cthrc1 in vivo does not affect bone mass (6). In their study, CtsK-Cre and Osx-Cre mice were crossed with mice carrying floxed Cthrc1 alleles to generate osteoclast and osteoblast specific deletion of Cthrc1 , respectively. Recent studies using CtsK-Cre mice revealed that Cre activity is not restricted to osteoclasts. Indeed, CtsK-Cre was shown to cause unexpected germline deletion of genes in mice (20). Additional concerns about the assumed osteoclast specificity of CtsK-Cre mediated recombination are also raised by a study demonstrating that Ctsk is expressed in mesenchymal progenitor cells, indicating that Ctsk-Cre mediated Cthrc1 deletion will not be restricted to osteoclasts but instead could also target progenitor cells differentiating into bone-forming cells (7).

There are also potential issues with regards to the use of Osx-Cre mice for expressing Cre selectively in osteoblasts, osteocytes and hypertrophic chondrocytes, because a recent study reported Osx expression also in stromal cells, adipocytes, and perivascular cells of bone marrow (21). Although the relationship between marrow fat and bone is increasingly appreciated (22-24), the potential influence of stromal cells and perivascular cells on bone is still largely unknown. Given that a role for CTHRC1 in adipogenic differentiation was identified(5), the unintended recombination activity mediated by Osx-Cre may make interpretation of a postnatal bone phenotype more difficult than originally anticipated.

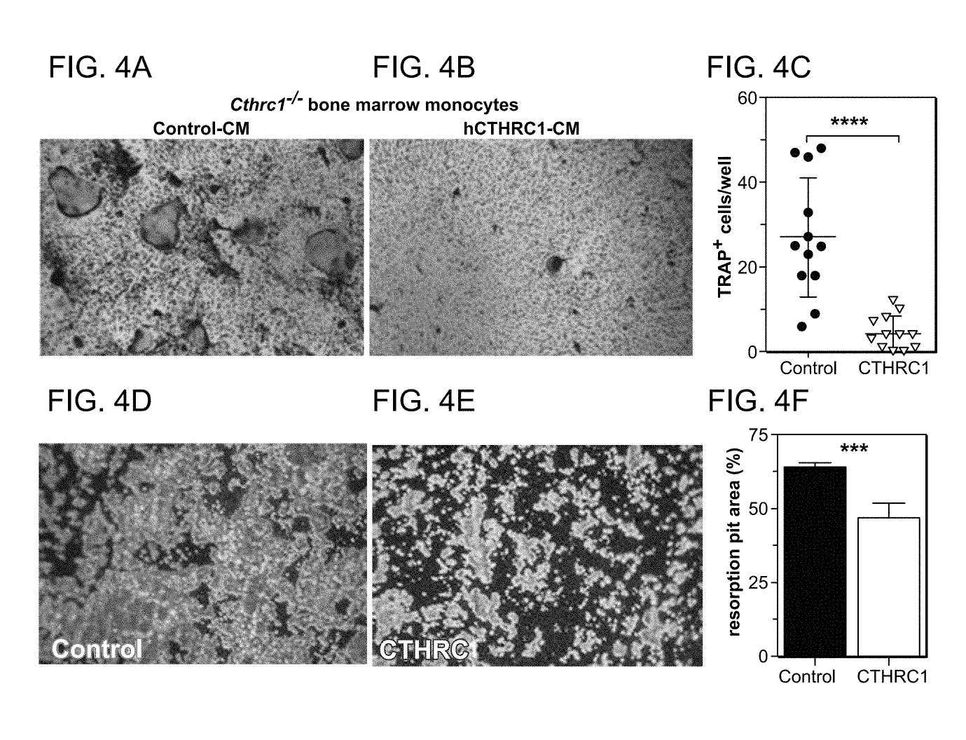

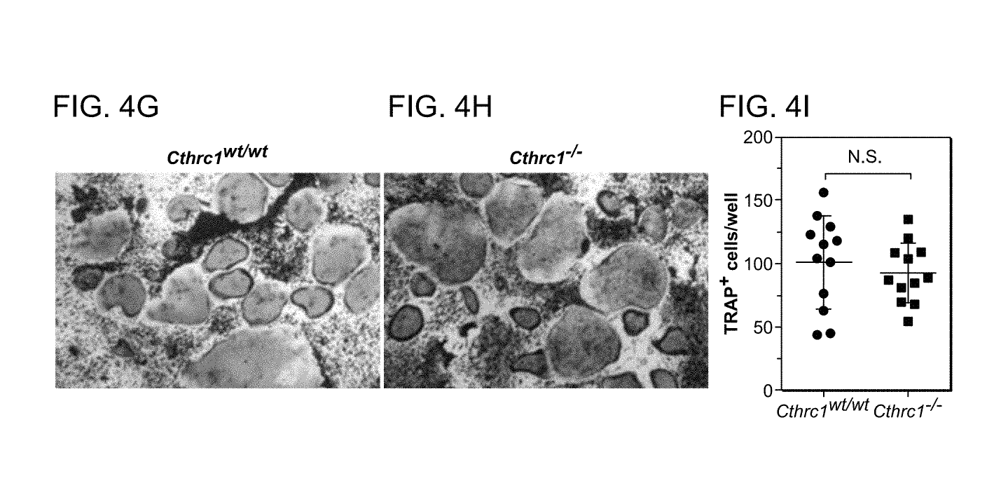

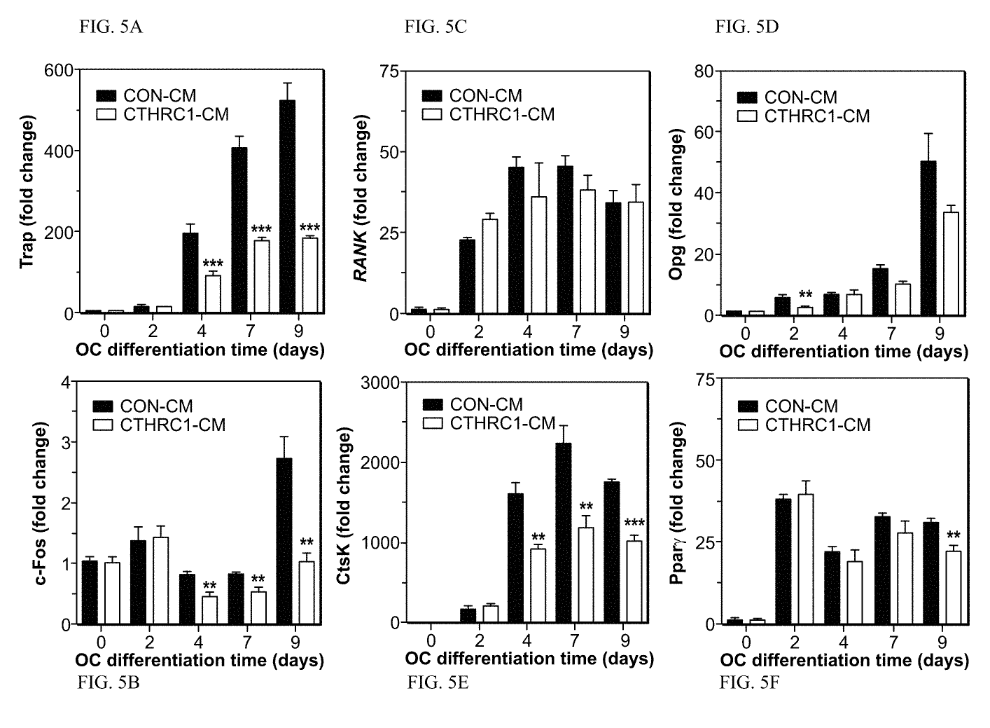

These findings of increased bone resorption and arthritis reveal a aspect for the function of CTHRC1 in bone homeostasis (FIG. 4A-C). In cultures of bone marrow-derived monocytes from Cthrc1 knockout mice, CTHRC1 exhibits strong inhibition of TRAP⁺ multinucleated osteoclast formation together with significant downregulation of osteoclastogenic gene expression and pit-forming bone resorption activity (FIG. 4D-I). Cthrc1 null cells can differentiate into osteoclasts as effectively as wildtype cells. These data support the in vivo findings and further support a role for CTHRC1 as a secretory protein from osteoblasts and osteocytes functioning as a key factor inhibiting osteoclastogenesis. In addition, CTHRC1 expression by activated fibroblast-like synoviocytes may have anti-inflammatory functions inhibiting arthritic joint destruction.

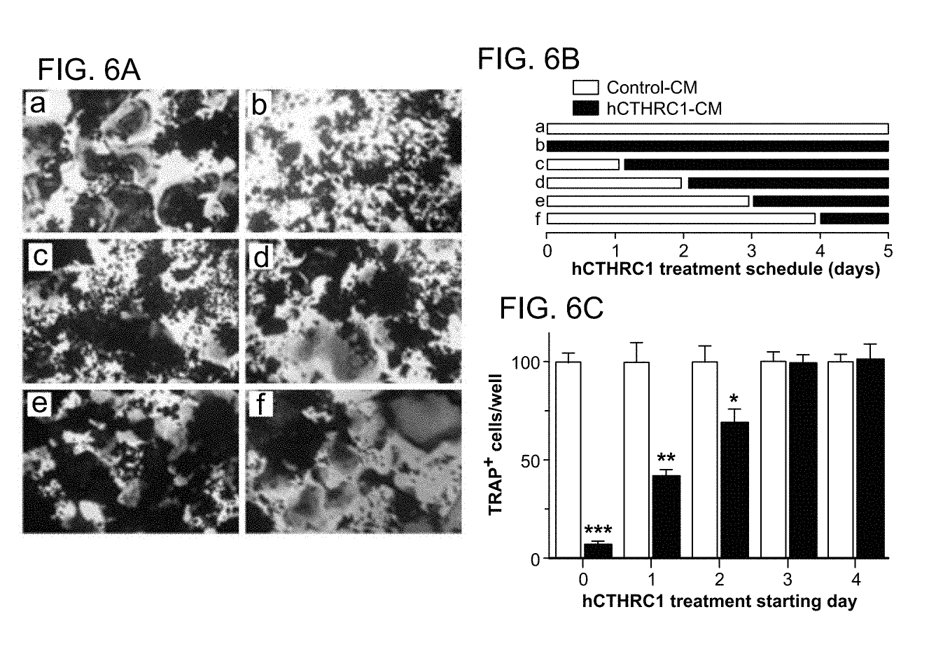

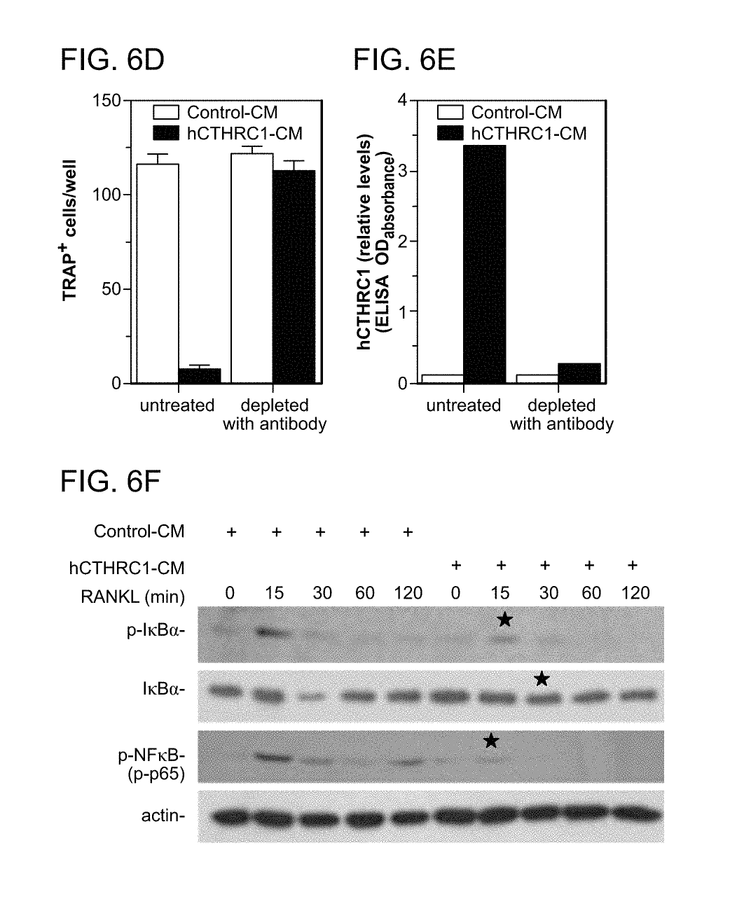

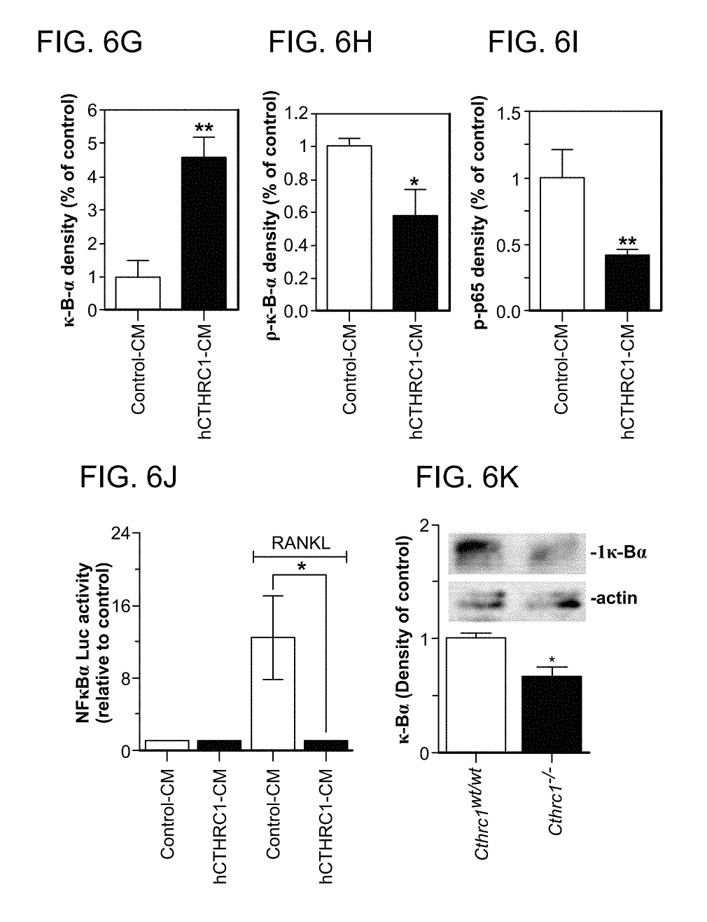

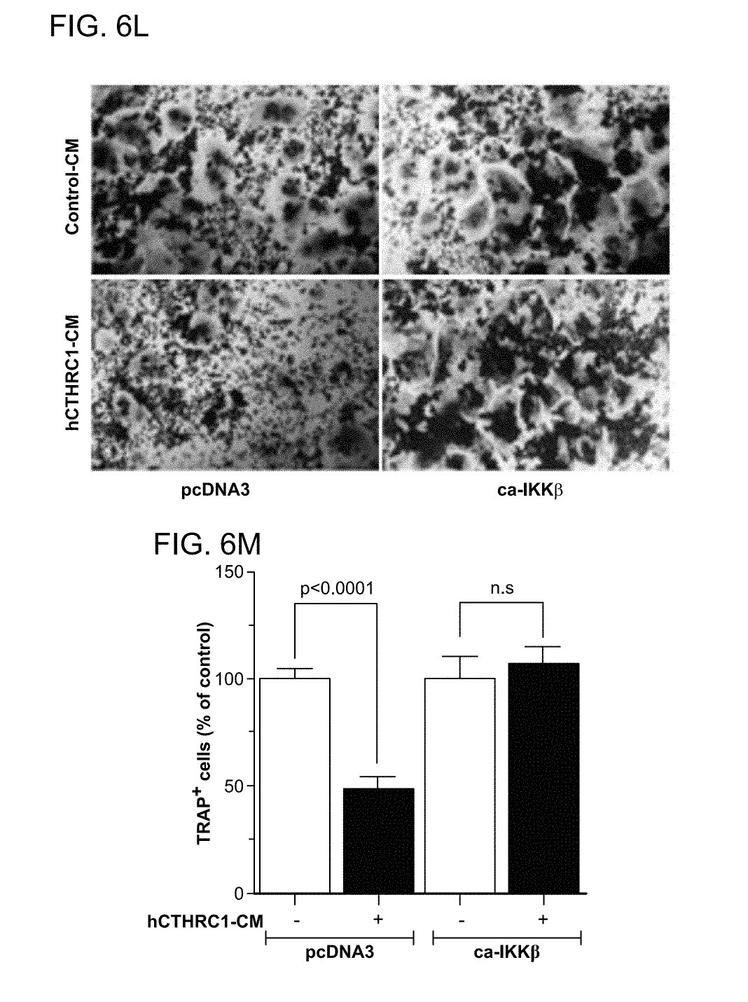

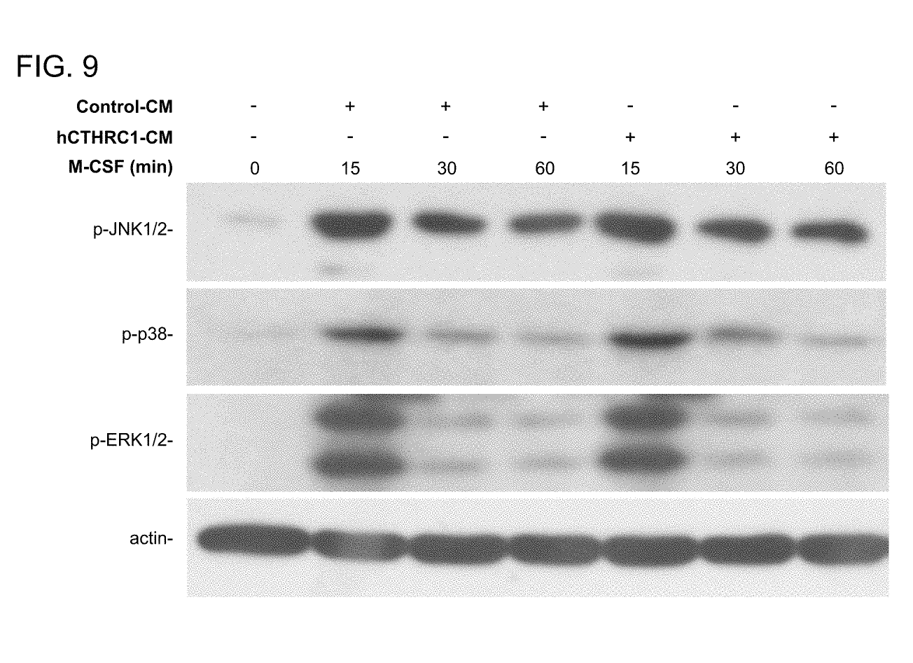

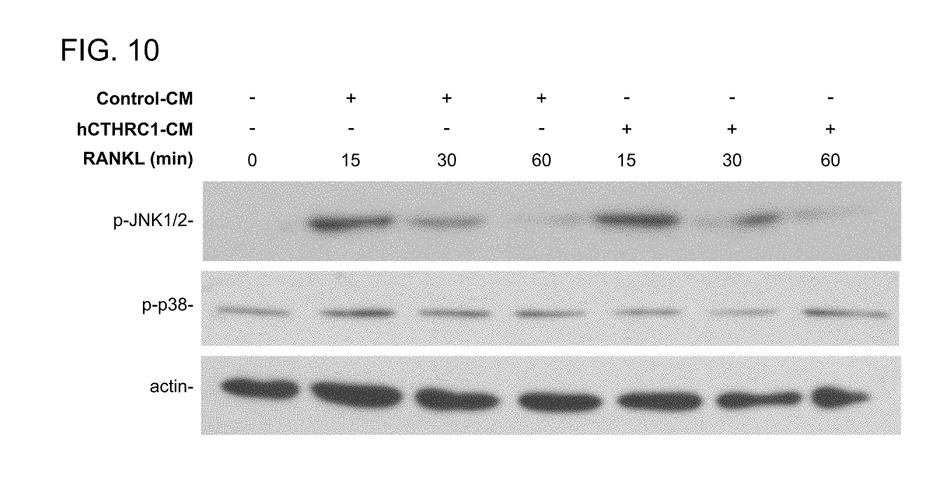

Osteoclast precursors originate from hematopoietic progenitor cells of the macrophage/monocyte lineage and the differentiation of these cells into bone-resorbing mature osteoclasts is a multistep process that involves cell proliferation, commitment, fusion, and activation (25,26). The time-course experiments indicate that CTHRC1 strongly inhibits RANKL-induced osteoclast differentiation only when added at an early stage of differentiation (FIG. 6), indicating that CTHRC1 may affect signaling events in osteoclast precursors. M-CSF and RANKL are two major factors essential for osteoclast differentiation. Immunoblotting data indicate that CTHRC1 does not affect M-CSF-stimulated early downstream signal transduction, such as ERK1/2, JNK and p38 (FIG. 9). With no effect of CTHRC1 on M-CSF-mediated cell proliferation, these findings indicate that CTHRC1 may exert its effect by modulating osteoclast differentiation. In support of this, it was found that CTHRC1 strongly inhibits RANKL-stimulated NFκB signaling activation by inhibiting phosphorylation and degradation of NFκBα without affecting the activation of JNK and p38 (FIG. 10). Although CTHRC1 did not affect early ERK1/2 activation after RANKL stimulation, it was associated with a significant reduction of ERK1/2 activation during later stages. ERK1/2 activation is known to be an essential step for transcriptional complex formation of AP-1 during RANKL-stimulated osteoclast formation; therefore, reduction of ERK1/2 activation by CTHRC1 may also contribute to its inhibition of osteoclast differentiation.

It has been reported that CTHRC1 can interact with Fzd and Ror2 to stabilize the WNT-receptor complex and activate the planar cell polarity pathway (27) through activation of Rac1 and RhoA. However, studies on bone from both Wnt5a and Ror2 mutant mice indicate that both have increased bone mass (28). In vitro studies indicate that the Wnt5a-Ror2 signaling axis plays a positive role in regulating osteoclast formation by enhancing RANK expression in osteoclast precursors (28) via activation of JNK. On the other hand, it has been reported that Rac1 acting upstream of TAK1 to induce NFκB activation is required for the normal differentiation of osteoclast precursors (29,30), whereas RhoA was reported to inhibit osteoclast differentiation (31). Bone phenotypes of Wnt5a and Ror2 mutant mice are the opposite of what is found in Cthrc1 null mice, and furthermore, the in vitro data indicate that CTHRC1 has no effect on RANK expression and JNK activation (FIG. 5) (28). This suggests that cell surface receptors mediating the biological effects of CTHRC1 have yet to be identified.

From a therapeutic point of view, as a secreted and circulating factor that inhibits osteoclastogenesis and immune responses, CTHRC1 may be a very attractive candidate to target conditions associated with low bone mass as well as arthritis.

### Collagen Triple Helix Repeat Containing-1 (Cthrc 1) Protein

Collagen triple helix repeat containing-1 (CTHRC1) is a protein isolated from a cDNA library of injured arteries. CTHRC1 functions as an inhibitor of TGF-β signaling. CTHRC1 is susceptible to cleavage by proteases and purified CTHRC1 forms aggregates, making it difficult to perform cell binding studies and protein interaction studies. Expression analyses of CTHRC1 in tissues have been performed by in situ hybridization, immunohistochemistry and RT-PCR analysis. CTHRC1 has also been found in plasma. CTHRC1 plasma levels in healthy human volunteers ranged from 16-440 ng/ml.

### Osteoporosis

Osteoporosis is a progressive bone disease characterized by a decrease in bone mass and bone density, which often leads to an increased risk of fracture. In osteoporosis, the bone mineral density (BMD) is reduced, bone microarchitecture deteriorates, and the amount and variety of proteins in bone are altered. Osteoporosis is defined by the World Health Organization (WHO) as a bone mineral density of 2.5 standard deviations or more below the mean peak bone mass (average of young, healthy adults) as measured by dual-energy X-ray absorptiometry. The term “established osteoporosis” includes the presence of a fragility fracture. The disease may be classified as primary type 1, primary type 2, or secondary. Primary type 1 or postmenopausal osteoporosis is most commonly seen in women after menopause. Primary type 2 osteoporosis or senile osteoporosis occurs after age 75 and is seen in both females and males at a ratio of 2:1. Secondary osteoporosis may arise at any age and affects men and women equally. This form results from chronic predisposing medical problems or disease, or prolonged use of medications such as glucocorticoids (steroid- or glucocorticoid-induced osteoporosis).

The diagnosis of osteoporosis can be made using conventional radiography and by measuring the bone mineral density (BMD; Guglielmi G and Scalzo G Diagnostic Imaging Europe, 26: 7-11, incorporated herein by rererence). The most popular method of measuring BMD is dual-energy x-ray absorptiometry. In addition to the detection of abnormal BMD, the diagnosis of osteoporosis requires investigations into potentially modifiable underlying causes; this may be done with blood tests.

### Pharmaceutical Compositions and Administration

The present invention comprises pharmaceutical preparations comprising a human CTHRC1 agonist (or antagonist) polypeptide together with a pharmaceutically acceptable carrier. Such compositions are useful for the treatment or prevention of fatty liver disease, low bone mass, and muscle weakness, or for the prevention or treatment of any one or more of the risk factors associated with these conditions. Polypeptides of the invention may be administered as part of a pharmaceutical composition. The compositions should be sterile and contain a therapeutically effective amount of the polypeptides in a unit of weight or volume suitable for administration to a subject.

As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal

The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient.

The therapeutic composition of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups also can be derived from inorganic bases such as, for example, sodium, potassium, ammonium, calcium or ferric hydroxides, and such organic bases as isopropylamine, trimethyl amine, 2-ethylamino ethanol, histidine, procaine and the like. Particularly preferred are the salts of TFA and HCl.

Physiologically tolerable carriers are well known in the art. Exemplary of liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes.

Liquid compositions also can contain liquid phases in addition to and to the exclusion of water. Exemplary of such additional liquid phases are glycerin, vegetable oils such as cottonseed oil, and water-oil emulsions.

A therapeutic composition contains an inflammation inhibiting amount or a fibrosis inhibiting amount of an Cthrc1 polypeptide of the present invention, typically formulated to contain an amount of at least 0.1 weight percent of Cthrc1 polypeptide per weight of total therapeutic composition. A weight percent is a ratio by weight of inhibitor to total composition. Thus, for example, 0.1 weight percent is 0.1 grams of inhibitor per 100 grams of total composition.

These compositions can be stored in unit or multi-dose containers, for example, sealed ampoules or vials, as an aqueous solution or as a lyophilized formulation for reconstitution. As an example of a lyophilized formulation, 10 mL vials are filled with 5 mL of sterile-filtered 1% (w/v) aqueous Cthrc1 polypeptide solution, and the resulting mixture can then be lyophilized. The infusion solution can be prepared by reconstituting the lyophilized material using sterile Water-for-Injection (WFI).

The compositions can be administered in effective amounts. The effective amount will depend upon the mode of administration, the particular condition being treated, and the desired outcome. It may also depend upon the stage of the condition, the age and physical condition of the subject, the nature of concurrent therapy, if any, and like factors well known to the medical practitioner. For therapeutic applications, it is that amount sufficient to achieve a medically desirable result.

The dosage ranges for the administration of the Cthrc1 polypeptide vary. In general, amounts are large enough to produce the desired effect in which disease symptoms of a metabolic syndrome are ameliorated. The dosage should not be so large as to cause adverse side effects. Generally, the dosage will vary with the age, condition, sex and extent of the disease in the patient and can be determined by one of skill in the art. The dosage also can be adjusted by the individual physician in the event of any complication.

A therapeutically effective amount is an amount sufficient to produce a measurable inhibition of symptoms of a condition (e.g., an increase in bone mass or a decrease in muscle weakness). Such symptoms are measured in conjunction with assessment of related clinical parameters.

A therapeutically effective amount of a polypeptide of this invention in the form of a polypeptide, or fragment thereof, is typically an amount of polypeptide such that when administered in a physiologically tolerable composition is sufficient to achieve a plasma concentration of from about 0.1 microgram (ug) per milliliter (mL) to about 200 ug/mL, or from about 1 ug/mL to about 150 ug/mL. In one embodiment, the plasma concentration in molarity is from about 2 micromolar (uM) to about 5 millimolar (mM) or from 100 uM to 1 mM Cthrc1 polypeptide. In other embodiments, the doses of polypeptide ranges from about 500 mg/Kg to about 1.0 g/kg (e.g., 500, 600, 700, 750, 800, 900, 1000 mg/kg).

The agents of the invention can be administered parenterally by injection or by gradual infusion over time. In other embodiments, agents are administered intravenously, intraperitoneally, intramuscularly, subcutaneously, intracavity, transdermally, topically, intraocularly, orally, intranasally, and can be delivered by peristaltic means. In one embodiment, a therapeutic compositions containing an agent of this invention are administered in a unit dose, for example. The term “unit dose” when used in reference to a therapeutic composition of the present invention refers to physically discrete units suitable as unitary dosage for the subject, each unit containing a predetermined quantity of active material calculated to produce the desired therapeutic effect in association with the required diluent; i.e., carrier, or vehicle.

The compositions are administered in a manner compatible with the dosage formulation, and in a therapeutically effective amount. The quantity to be administered and timing depends on the patient to be treated, capacity of the patient's system to utilize the active ingredient, and degree of therapeutic effect desired. Precise amounts of active ingredient required to be administered depend on the judgement of the practitioner and are peculiar to each individual. However, suitable dosage ranges for systemic application are disclosed herein and depend on the route of administration. Suitable regimes for administration also are variable, but are typified by an initial administration followed by repeated doses at one or more hour intervals by a subsequent injection or other administration. Alternatively, continuous intravenous infusion sufficient to maintain concentrations in the blood in the ranges specified for in vivo therapies are contemplated.

The amount and frequency of administration of antibody would depend on a number of factors including, but not limited to, the condition to be treated.

### Therapy

As demonstrated herein, CTHRC1 inhibitors are useful for the treatment or prevention of low bone mass (i.e., osteoporosis). Subjects suffering, suspected of suffering, or prone to these conditions can be tested and monitored for expression levels of CTHRC1. Determining CTHRC1 levels can be performed at a single time point, or CTHRC1 levels can be monitored over time, as are many diagnostic markers, and substantial changes in CTHRC1 levels can be an indication that further testing for a metabolic disorder should be performed. Testing can be done using any assay specific for CTHRC1, for example any immunoassay, preferably an assay that is amenable to high throughput and/or automated screening methods. Antibodies to various portions of CTHRC1 can be generated using routine methods. Methods of epitope selection, antigen preparation, and antibody production are well known to those of skill in the art.

Therapy may be provided wherever therapy for these conditions is performed: at home, the doctor's office, a clinic, a hospital's outpatient department, or a hospital. Treatment generally begins at a hospital so that the doctor can observe the therapy's effects closely and make any adjustments that are needed. The duration of the therapy depends on the kind of disease being treated, the age and condition of the patient, the stage and type of the patient's disease, and how the patient's body responds to the treatment. Drug administration may be performed at different intervals (e.g., daily, weekly, or monthly). Therapy may be given in on-and-off cycles that include rest periods so that the patient's body has a chance to build healthy new cells and regain its strength.

A CTHRC1 inhibitor may be administered within a pharmaceutically-acceptable diluent, carrier, or excipient, in unit dosage form. Conventional pharmaceutical practice may be employed to provide suitable formulations or compositions to administer the compounds to patients suffering from a disease that is associated with a metabolic syndrome. Administration may begin before the patient is symptomatic. Any appropriate route of administration may be employed, for example, administration may be topical, parenteral, intravenous, intraarterial, subcutaneous, intratumoral, intramuscular, intracranial, intraorbital, ophthalmic, intraventricular, intrahepatic, intracapsular, intrathecal, intracisternal, intraperitoneal, intranasal, aerosol, suppository, or oral administration. For example, therapeutic formulations may be in the form of liquid solutions or suspensions; for oral administration, formulations may be in the form of tablets or capsules; and for intranasal formulations, in the form of powders, nasal drops, or aerosols.

Methods well known in the art for making formulations are found, for example, in “Remington: The Science and Practice of Pharmacy” Ed. A. R. Gennaro, Lippincourt Williams & Wilkins, Philadelphia, Pa., 2000. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for modulatory compounds include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

The formulations can be administered to human patients in therapeutically effective amounts (e.g., amounts which prevent, eliminate, or reduce a pathological condition) to provide therapy for a disease or condition. “Therapeutically effective amount” is intended to include an amount of a compound useful in the present invention or an amount of the combination of compounds claimed, e.g., to treat or prevent the disease or disorder, or to treat the symptoms of the disease or disorder, in a host. The combination of compounds is preferably a synergistic combination. Synergy, as described for example by Chou and Talalay, Adv. Enzyme Regul. 22:27-55 (1984), occurs when the effect of the compounds when administered in combination is greater than the additive effect of the compounds when administered alone as a single agent. In general, a synergistic effect is advantageously demonstrated at suboptimal concentrations of the compounds. Synergy can be in terms of lower cytotoxicity, increased activity, or some other beneficial effect of the combination compared with the individual components. The preferred dosage of a CTHRC1 inhibitor is likely to depend on such variables as the type and extent of the disorder, the overall health status of the particular patient, the formulation of the compound excipients, and its route of administration. If desired, treatment with an agent of the invention may be combined with therapies for the treatment of a metabolic syndrome.

For any of the methods of application described above, an agent of the invention of the invention is desirably administered intravenously or is applied to the tissue affected by metabolic syndrome (e.g., by injection).

### Kits

The invention provides kits for the treatment or prevention of a metabolic syndrome. In one embodiment, the kit includes a therapeutic or prophylactic composition containing an effective amount of an agent described herein. In some embodiments, the kit comprises a sterile container that contains a therapeutic or prophylactic composition; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLE 1

### Materials and Methods

Reagents and antibodies

Penicillin, streptomycin, and a-minimum essential medium (α-MEM) were obtained from Mediatech (Herndon, Va.), and fetal bovine serum (FBS) was obtained from Atlanta Biologicals. Soluble, recombinant murine M-CSF and human recombinant RANKL were obtained from PeproTech (Rocky Hill, N.J.) and R&D Systems (Minneapolis, Minn.). Anti-phospho-ERK1/2, anti-IκBα, anti-phospho-IκBα, anti-phospho-p65 (p-NFκB) and anti-cleaved caspase-3 antibodies were from Cell Signaling Technology, Inc. (Beverly, Mass.). Anti-mouse and anti-rabbit HRP conjugated IgGs were purchased from Jackson ImmunoResearch Laboratories, Inc (West Grove, Pa.). Tartrate-resistant acid phosphatase (TRAP) immunostaining was performed with biotinylated monoclonal anti-TRAP (1:500, clone ACPS/1070, Novus Biologicals). Apoptosis of calvarial osteoblasts derived from Cthrc1 null and wildtype mice was assessed by immunocytochemistry for activated caspase-3. Rabbit and mouse monoclonal anti-CTHRC1 antibodies were previously characterized and described (mmcri.org/antibody) (2).

Rabbit monoclonal anti-Cthrc1 clone Vli-55 (mmcri.org/antibody) was used for Western blotting and immunohistochemistry on paraffin-embedded, formalin-fixed tissues as described (3). For Western blotting of mouse tissue lysates for CTHRC1, the Avidin/Biotin Blocking Kit (Vector, Burlingame, Calif.) was used, followed by incubation with biotinylated Vli-08G09 (10 ng/mL) using the EZ-Link Biotinylation Kit (LC spacer) from Thermo Scientific (Rockford, Ill.), followed by incubation with Streptavidin-HRP (Vector, Burlingame, Calif.).

Unmodified human CTHRC1 (hCTHRC1) was produced in CHO-K1 cells by adenovirus transduction as described (3). Prior evidence suggested that C-terminally tagged CTHRC1 may interfere with function and therefore only wildtype hCTHRC1 was used in the form of serum-free conditioned medium (hCTHRC1-CM) added to the culture medium (60% final). All hCTHRC1-CM used was tested for hCTHRC1 concentration by ELISA (2) with hCTHRC1 levels consistently in the 100 ng/mL range. Serum-free conditioned medium from CHO-K1 cells transduced with a beta-galactosidase expressing adenovirus was used as a control. For experiments using hCTHRC1-depleted hCTHRC1-CM, the conditioned medium was incubated with monoclonal anti-hCTHRC1 IgGs Vli-13E09 and Vli-10G07 (1 μg/mL each, 3 hours at 4° C.), followed by removal of the hCTHRC1/antibody complexes with Protein A Sepharose (CL-4B, Amersham) chromatography.

### Mice

All protocols involving animals were approved by the Institutional Animal Care and Use Committee of the Maine Medical Center (protocol number 1505) and were in compliance with all applicable regulations and guidelines including the National Institutes of Health Guide for Care and Use of Laboratory Animals. Cthrc1 null mice were derived from matings of homozygous Cthrc1^{tm1Vli} mutants on the C57BL/6J background as described previously (3). C57BL/6J wildtype mice were used as controls. Mice were fed a standard rodent diet (Harlan, 2018 Teklad Global 18% Protein Rodent Diet) and water ad libitum, and housed with dry cellulose bedding (Harlan, 7070 Diamond) under a 14-hour daylight-10 hour night cycle.

### Bone Analyses

To evaluate bone volume and architecture by micro-computed tomography (μCT), mouse tibiae were fixed in 70% ethanol and scanned using a Scanco μCT instrument (Scanco Medical) at several resolutions for both overall tibial assessment and structural analysis of trabecular and cortical bone. Trabecular bone parameters were calculated using the Scanco software to analyze the bone scans from the trabecular region directly distal to the proximal tibial growth plate. Bone histomorphometric analyses were conducted in the Baron laboratory on non-decalcified sections as previously described (8,9). Calcein (Sigma/Aldrich, 20 mg/kg injected 7 days before euthanasia) and demeclocycline (Sigma/Aldrich, 30 mg/kg injected 2 days before euthanasia) were used to label bone. Terminology and units follow the recommendations of the Histomorphometry Nomenclature Committee of the American Society for Bone and Mineral Research (10,11).

### Mechanical Testing

Femurs were mechanically tested in three-point bending using an electromechanical materials testing machine (Synergie 100, MTS Systems, Eden Prairie, Minn.) as described (12).

### Western Blotting

Western blotting of bone and marrow tissue lysates was performed as previously described using primary antibodies (anti-Cthrc1, 10 ng/ml; anti-actin, 1:1000) followed by secondary antibodies (1:5000) conjugated with horseradish peroxidase (HRP) (2). Marrow was separated from bone by blowing it out of the shaft with a pipet.

Quantitative evaluation of immunoblots was performed by densitometry using ImageJ software.

### DNA Transfection and Luciferase Reporter Assays

An expression vector for constitutively active IKKβ (Addgene, IKK-2 S177E S181E) or pcDNA3.0 was used to transfect RAW264.7 cells with Fugene 6 (Roche) transfection reagent in OPTM-1 medium, which was replaced with osteoclastogenic differentiation medium 24 hours later. For the reporter assays, the Dual-Luciferase Reporter Assay (Promega) was used with RAW264.7 cells cotransfected with luciferase reporter plasmids (NFκB-Luc and AP1-Luc, PathDetect, Agilent Technologies). 24 hours later the cells were stimulated with differentiation medium containing RANKL (100 ng/mL) in the presence of control-CM or hCTRHC1-CM before harvesting cell lysates for analyses 24 hours later. All assays were performed in replicates of ≥3.

### Quantitative RT-PCR

The methods for this were previously described (5). All primer sequences are listed in Table 3.

TABLE 3
Sequences of primers used for PCR amplification.

| | Forward Primer | Reverse Primer |
|---|---|---|
| Nfatc1 | TCATCCTGTCCAACACCAAAGTC | ATGTGAACTCGGAAGACCAGCC |
| | (SEQ ID NO: 4) | (SEQ ID NO: 5) |
| Ctsk | GGCATCTTTCCAGTTTTACAGCAG | GGCGTTGTTCTTATTCCGAGC |
| | (SEQ ID NO: 6) | (SEQ ID NO: 7) |
| c-Fos | CGAAGGGAACGGAATAAGATGG | AGACCTCCAGTCAAATCCAGGG |
| | (SEQ ID NO: 8) | (SEQ ID NO: 9) |
| Trap | TTCCAGGAGACCTTTGAGGACG | GAGTTGCCACACAGCATCACTG |
| | (SEQ ID NO: 10) | (SEQ ID NO: 11) |
| Opg | GCACAGTGAGGAGGAAGACATTG | ACCTGAGAAGAACCCATCTGGAC |
| | (SEQ ID NO: 12) | (SEQ ID NO: 13) |
| Rankl | ACACCTCACCATCAATGCTGCC | TTCGTGCTCCCTCCTTTCATC |
| | (SEQ ID NO: 14) | (SEQ ID NO: 15) |
| Pparg | TTTCAGAAGTGCCTTGCTGTGGGG | GATTTGTCCGTTGTCTTTCCTGTC |
| | (SEQ ID NO: 16) | (SEQ ID NO: 17) |
| Rank | CATCGTTCTGCTCCTCTTCATCTC | CTTCACACACTTCTTGCTGACTGG |
| | (SEQ ID NO: 18) | (SEQ ID NO: 19) |
| Gapdh | ACACATTGGGGGTAGGAAC | AACTTTGGCATTGTGGAAGG |
| | (SEQ ID NO: 20) | (SEQ ID NO: 21) |
| Cthrc1 | CCAGGTCGGGATGGATTC | AGCGTCTCCTTTGGGGTAAT |
| | (SEQ ID NO: 22) | (SEQ ID NO: 23) |

### Osteoclastogenic Differentiation

Osteoclasts were differentiated from mouse bone marrow monocytes as described elsewhere (13). Briefly, bone marrow cells were purified with a 40 μm cell strainer to remove mesenchymal cells, cultured with 40 ng/mL of M-CSF in α-MEM containing 10% FBS for 3 days until cells reached confluence. Then the cells were differentiated for 9 days with M-CSF (40 ng/mL) and RANKL (100 ng/mL) in the presence of serum-free conditioned medium harvested from control transfected CHO-K1 cells (Control-CM), CHO-K1 cells stably transfected with a beta-galactosidase-(Control-CM) or hCTHRC1 adenovirus transduced CHO-K1 cells (hCTHRC1-CM) and 10% FBS. At day 9, the culture was terminated, and cells were then fixed with 2.5% glutaraldehyde (Sigma). Osteoclasts were identified by tartrate-resistant acid phosphatase staining kit (Sigma/Aldrich). Mature osteoclasts were identified as multinucleated (>3 nuclei) TRAP⁺ cells.

RAW264.7 cells were cultured in α-MEM containing 10% FBS. Osteoclastogenic differentiation was induced by addition of RANKL (100 ng/mL) to the culture medium. Differentiation was allowed to continue for 4-5 days with media changed every 2 days.

### Osteoblastic Cell Culture and Assays

Osteoblasts were differentiated from mouse bone marrow stromal cells and calvarial osteoblasts as described (13). To culture osteoblasts, bone marrow was harvested from femurs and tibiae of wildtype and Cthrc1 null mice (n=3). Bone marrow cells were dispersed by passage through a 25G needle 5 times. Cells were plated in 12-well plates (5×10⁶ cells/well) in plating medium (α-MEM containing 10% FBS) and cultured for 3 days before cells reached confluence. Primary calvarial osteoblasts were isolated from newborn calvaria by sequential digestion with 0.42 mg/mL collagenase P (Roche) and trypsin (0.5%, Gibco).

Primary osteoblastic cells from the second to fourth fraction were pooled and used for osteogenic differentiation. To induce osteogenic differentiation, α-MEM containing 10% FBS, β-glycerophosphate (8 mM) and ascorbic acid (50 μg/mL) was used and the medium was replaced every other day over a 21 day period. Early osteogenic differentiation was assessed by alkaline phosphatase histochemistry (86R-1KT, Sigma-Aldrich), whereas mature bone formation was determined by Alizarin red staining and von Kossa staining as described.

### Colony-Forming Assay

To determine potential effects of CTHRC1 on bone marrow stromal cells and bone marrow derived monocyte proliferation, colony-forming assays were performed by seeding 0.5×10⁶ cells/mL from Cthrc1 null mice in 6 well tissue culture plates in growth medium as described above, supplemented with either control LacZ conditioned-medium or CTHRC1 conditioned-medium. After four days of culture, the culture medium was removed and the cells were fixed with 4% paraformaldehyde for 10 min, and cell colonies were stained using crystal violet.

### Bone Resorption Assay

Bone resorption was assessed by culturing bone marrow cells in Osteo Assay Surface 96-well plates (Corning Life Sciences, Lowell, Mass., USA) as previously described (14). In brief, bone marrow monocytes isolated from Cthrc1 null mice were cultured with differentiation medium containing control LacZ conditioned-medium or CTHRC1 conditioned-medium as described above. Differentiation media was changed every 3 days until the end of a 17-day culture period. After wells were washed with PBS, 150 μL of 10% sodium hypochlorite was added to each well and incubated for 5 min. Wells were washed twice with water, and stained with a modified von Kossa stain (5% [w/v] aqueous silver nitrate solution) in the dark for 30 min. After staining, wells were soaked in water for 5min, and treated with 100 μL of 5% sodium carbonate (w/v in 10% formalin). Incubating for 5 min at room temperature reduces ionic silver to metallic silver, and the unresorbed mineralized surface turns black, whereas the resorbed areas are white. Digital images were captured and the percentage of resorbed area/well was quantified using ImageJ software. Eight wells were assessed per treatment.

### Collagen Antibody-Induced Arthritis Model

Female homozygous Cthrc1^{tmlGVli} mutants on the C57BL/6J background and wildtype C57BL/6J controls (n=4 per group, 10-12 weeks of age) were injected intraperitoneally with the anti-collagen II antibody cocktail (Chondrex Inc., catalog #53010, 5 mg/mouse). Two days later all mice received an intraperitoneal injection of lipopolysaccharide (Chondrex Inc., catalog #9028, 50 μg/mouse). Arthritis was scored in a blinded fashion on days 7, 10, 14, and 21 after the antibody injection. Score 0: normal; score 1: mild but definite redness and swelling of the ankle or front paw, or apparent redness and swelling limited to individual digits, regardless of the number of affected digits; score 2: moderate redness and swelling of ankle or front paw; score 3: severe redness and swelling of the entire paw including digits; score 4: maximally inflamed limb with involvement of multiple joints. Mice were euthanized 21 days after the antibody injection for analysis of bones by histology. Histology scoring of arthritis combined for ankle and knee joints was performed in a blinded manner by scoring the amount/area of inflammatory cells on a scale from 0 (no) to 4 (maximal) inflammation. Bone erosion and pannus infiltrating the calcaneus was quantified by image analysis measuring the area of eroded bone and pannus surrounding the cortical bone of the calcaneus. The Mann-Whitney U test (GraphPad Prism) was used to assess statistical significance.

### Statistical Analyses

Statistical analyses were performed in GraphPad Prism (Version 5, GraphPad Software, Inc., San Diego, Calif.). Means of data for Cthrc1 null mice and wildtype mice were compared using Student's t-test. Values for p<0.05 were considered statistically significant. With the expected differences between groups, it was calculated that a sample size of 6 in each group (unless stated otherwise) would have 80% power (p=0.05) to detect a significant difference in means for the bone parameters. In the collagen antibody-induced arthritis model 80% power was achieved with n=4 mice per group.

### EXAMPLE 2

### CTHRC1 is Expressed in Bone-Forming Cells

To determine where CTHRC1 is expressed in bone immunohistochemistry, western blotting and RT-PCR was performed to identify the cell types and compartments of femurs that express CTHRC1 Immunoreactive CTHRC1 localized specifically to osteoblasts lining bone surfaces, whereas adjacent multinucleated osteoclasts did not express CTHRC1 (FIG. 1A). TRAP expression characteristic for osteoclasts revealed no overlap with expression of CTHRC1 as analyzed by confocal microscopy of double immunolabeling (FIG. 1B). CTHRC1 was also expressed by some but not all osteocytes where it localized to osteocyte processes of individual cells (FIG. 1C-E. arrows). CTHRC1 immunoreactivity within the osteocyte canaliculi is suggestive of CTHRC1 secretion into the extracellular space (FIG. 1C-F). Complete absence of any staining performed on femur sections from Cthrc1 null mice confirmed specificity of the immunostaining performed with the monoclonal antibody (FIG. 1G). RT-PCR and Western blotting were used to confirm the immunohistochemistry results. RNA isolated from bone marrow, cortical bone (midshaft) and trabecular bone (proximal and distal epiphysis) showed expression of the Cthrc1 transcript in the bone fractions but not the marrow (FIG. 1H). It was also determined Cthrc1 expression during osteogenic differentiation of bone marrow stromal cells (BMSCs) and found that these cells express Cthrc1 with transcript levels increasing as cells differentiate (FIG. 1I). Western blot analysis of lysates prepared separately from the bone and marrow fraction further confirmed the absence of CTHRC1 in marrow cells (FIG. 1J). The biotinylated monoclonal antibody showed no CTHRC1 band in samples from Cthrc1 null mice (FIG. 1G). These results indicate that CTHRC1 in bone is derived from osteoblasts and osteocytes but not osteoclasts.

### EXAMPLE 3

### Bone Formation is Altered in Cthrc1 Null Mice

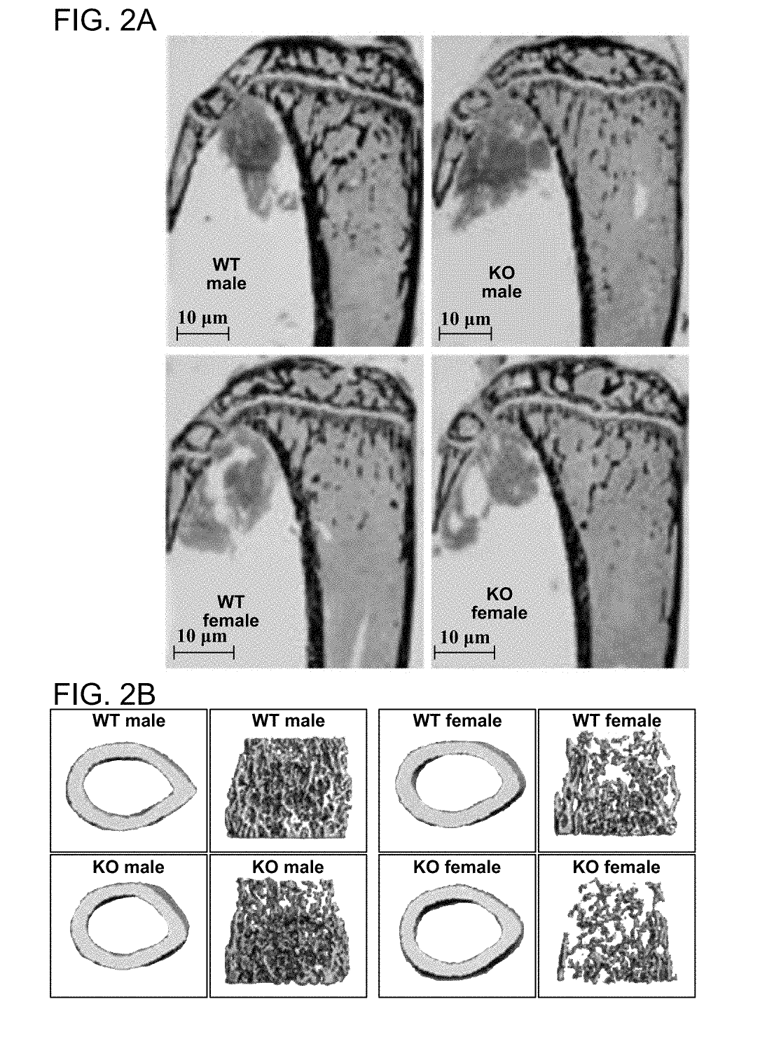

Previous studies indicated that CTHRC1 affects bone formation (4,6). Unlike previous studies, the Cthrc1 null mice analyzed here were on a pure C57BL/6J genetic background. The targeting strategy generated a global null mutant that eliminated the potential confounding effects of circulating CTHRC1 expressed in tissues other than bone (2,3). In addition, bones from males and females were analyzed. The skeletal phenotype of 8 week old Cthrc1 knockout mice was analyzed by histology, histomorphometry and μCT performed on non-decalcified specimens. As shown in Table 1 and FIG. 2, Cthrc1^{tm1Vli} mice clearly show lower trabecular bone mass compared to wildtypes in both genders. All the structure parameters [Bone Volume (BV/TV), Trabecular Thickness (Tb.Th), Trabecular Number (Tb.N) and Trabecular Separation (Tb.Sp)] in female Cthrc1 null mice showed significant differences, and the same trends occurred in male mice, even though changes in some parameters did not reach significance. To understand the mechanism that led to lower trabecular bone mass in these Cthrc1^{tm1Vli} mice, the dynamic and cellular parameters of bone remodeling were assessed. The dynamic parameters of bone formation [MAR (Mineral Apposition Rate), Bone Formation Rate (BFR)] showed significant decreases in Cthrc1 null mice compared to wildtypes (for example, BFR/BS was decreased almost 30% in male and 45% in female). It is important to note here that most of the decrease in bone formation rate is attributable to a profound decrease in MAR, indicating a decrease in the ability of individual osteoblasts to produce bone matrix. These findings were supported by the changes observed in osteoblast parameters. The Number of Osteoblasts (N.Ob/B.Pm) and Osteoblast Surface (Ob.S/B.Pm) were significantly decreased in male Cthrc1 null mice. The same trend was seen in female mice, but did not reach statistical significance (25% lower N.Ob/B.Pm and 30% lower Ob.S/B.Pm), suggesting that in addition to a decrease in individual osteoblast activity there is also a decrease in the number of these cells. In agreement with Mineralizing Surface (MS) and MAR, Osteoid Surface (OS/BS) and Osteoid Thickness (O.Th) showed a significant decrease in both genders, confirming that Cthrc1 null mice exhibit a defect in bone formation.

### EXAMPLE 4

### Effects of Cthrc1 on Mechanical Properties of Bone

Three-point bending tests and μCT were performed to compare the mechanical and architectural properties of bones from 8 week old male and female wildtype and Cthrc1 null mice on the C57BL/6J background. μCT was performed on the mid-diaphysis of the femur to measure cortical architecture and mineral density. As shown in Table 2 and FIG. 2, female Cthrc1 null mice had similar cortical architecture, and only a slight increase (not statistically significant) in cortical tissue mineral density relative to the wildtypes. In contrast, male Cthrc1 null mice had worse cortical bone properties, including greater cortical porosity and lower femur length, cortical thickness, cortical tissue mineral density, cortical area, total area, cortical bone area fraction, and the maximum, minimum, and polar moments of inertia relative to the wildtype male mice. These μCT results predict that deletion of Cthrc1 would have a negative effect on femoral biomechanical properties in male mice.

Three-point bending of the femoral diaphysis showed very little difference between the mechanical properties of the femurs from wildtype and Cthrc1 null female mice, but, as predicted by the inferior bone morphology, there were significant deficits in Cthrc1 null male mice. Specifically, male Cthrc1 null mice had 29% lower maximum moment and 32% lower bending stiffness relative to wildtype mice (Table 2). While there was a decrease in bending stiffness in the Cthrc1 null male mice, there was no difference in bending modulus, suggesting that the differences in biomechanical properties between the Cthrc1 null and wildtype male mice are due to cortical bone architecture rather than the material properties of the bone. The bones of Cthrc1 null male mice also required significantly less work (energy) to cause them to yield and fracture.

In summary, deletion of Cthrc1 has minimal effects on cortical bone properties in female mice. In contrast, male Cthrc1 null mice have shorter femurs, with reduced cross-sectional area and cortical morphology, leading to worse biomechanical properties compared to wildtype mice.

### EXAMPLE 5

### CTHRC1 Does Not Affect Osteogenic Differentiation of BMSCs In Vitro

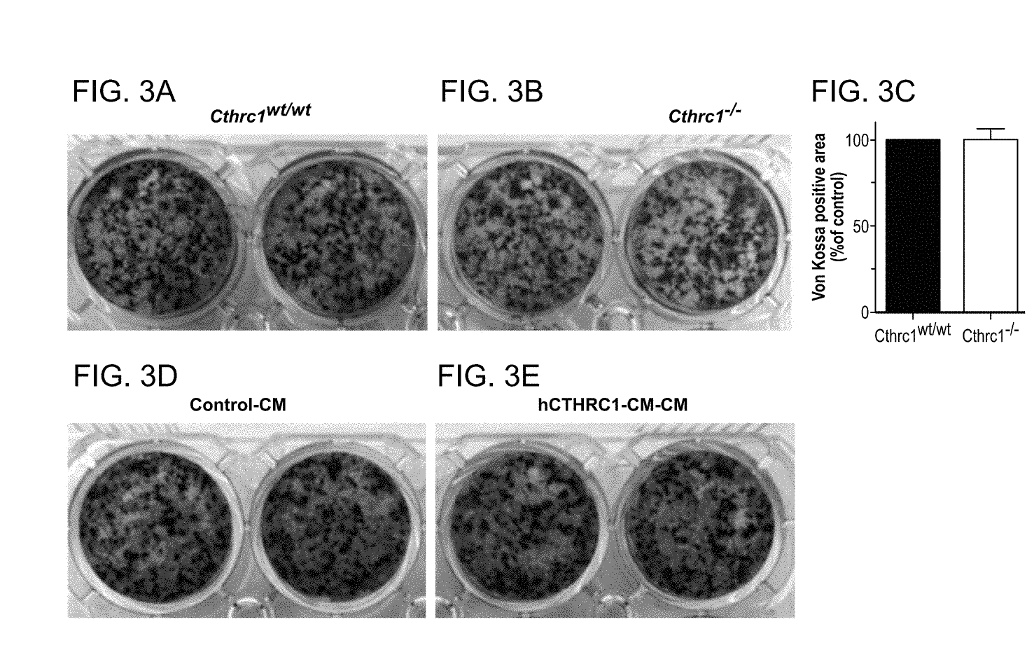

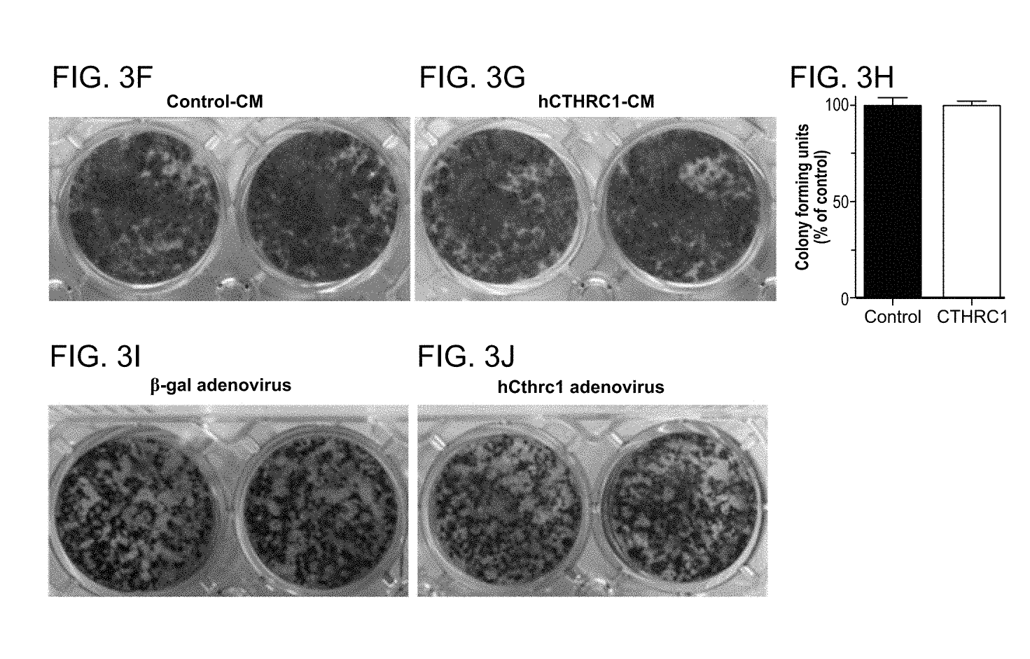

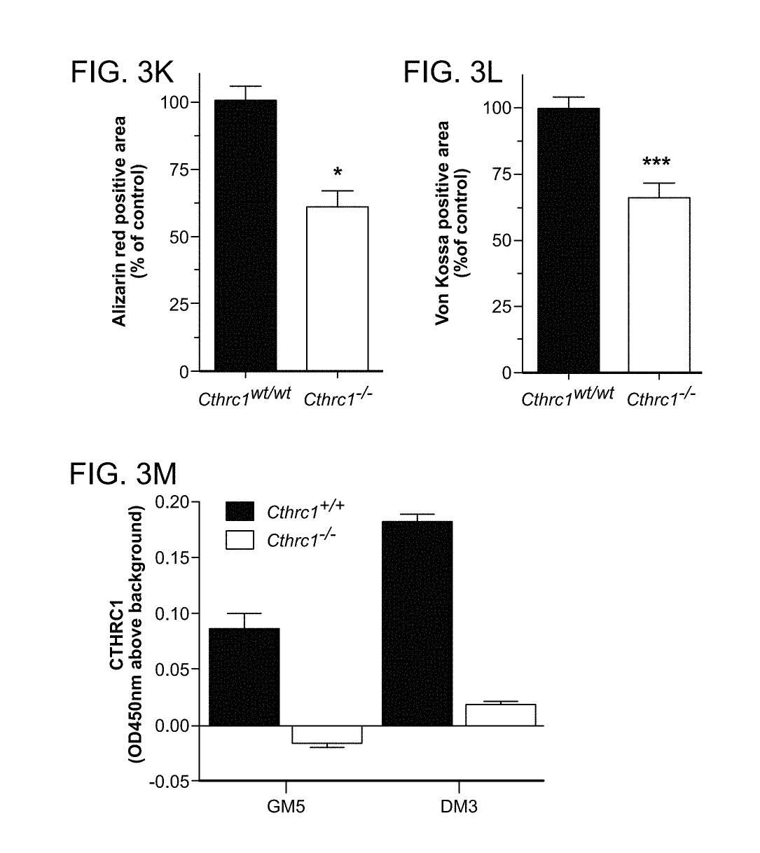

To determine if CTHRC1 has a cell-autonomous effect on osteogenic differentiation of BMSCs, these cells were isolated from both wildtype and Cthrc1 null mice and induced osteogenic differentiation by adding ascorbic acid and β-glycerophosphate. In contrast to previous reports (4,6), it was found that BMSCs isolated from Cthrc1 knockout mice can differentiate into osteoblasts as effectively as wildtype BMSCs as determined by Alizarin red and von Kossa staining (FIG. 3A-C). In addition, when BMSCs derived from Cthrc1 null mice are differentiated in the presence of hCTHRC1 no difference between control-treated cells and hCTHRC1-treated cells with respect to osteogenic differentiation as determined by Alizarin red (FIG. 3D, E) and alkaline phosphatase staining (FIG. 3F, G) is identified. Proliferation of bone marrow stromal cells was not impacted by the presence or absence of CTHRC1 with similar numbers of colonies formed in both treatment groups (FIG. 3H). Osteogenic differentiation in Cthrc1 knockout BMSCs after transduction with an hCTHRC1 adenovirus or control adenovirus (FIG. 3I, J) and observed no effect on osteogenic differentiation was also performed. Therefore, the data indicate that for BMSCs growing in vitro, there is no cell-autonomous effect of CTHRC1 in regulating osteogenic differentiation.

### EXAMPLE 6

### CTHRC1 Promotes Osteogenic Differentiation of Calvarial Osteoblasts In Vitro

To determine whether the effects of CTHRC1 on osteogenic differentiation are cell type-dependent, osteogenic differentiation also of calvarial osteoblasts derived from both Cthrc1 null and wildtype mice was examined Although early osteogenic differentiation as assessed by alkaline phosphatase activity was similar among cultures, late stage differentiation markers were significantly reduced in Cthrc1 null osteoblast cultures (FIG. 3K, L). It was verified that osteoblast cultures were indeed expressing CTHRC1 by measuring CTHRC1 levels in the medium by ELISA while cells were in growth medium and differentiation medium (FIG. 3M, GM5, DM3). CTHRC1 was secreted into the medium by wildtype osteoblasts but not by Cthrc1 null cells. These data indicate that CTHRC1 has cell-autonomous functions in calvarial osteoblasts.

### EXAMPLE 7

### Bone Resorption is Increased in Cthrc1 Null Mice

Next, bone resorption parameters, osteoclast number (N.Oc/B.Pm) and osteoclast surface (Oc.S/B.Pm), were examined in wildtype and Cthrc1 null mice. In contrast to previous reports, it was observed that both parameters were significantly increased by approximately 41% and 47% in the male Cthrc1 null mice, respectively (Table 1). In female mice, the osteoclast number in Cthrc1 null mice is also significantly increased by approximately 36% when compared with wildtype mice (Table 1). Although the osteoclast surface was also increased in female Cthrc1 null mice, it did not reach statistical significance (Table 1). Importantly, the Eroded Surface (ES/BS) was significantly increased in both genders (Table 1). These data clearly indicate that the lower trabecular bone mass observed in Cthrc1 knockout mice is also the result of increased bone resorption activity, in addition to the decrease in bone formation. These data indicate that the lower trabecular bone mass observed in Cthrc1 null mice is also the result of increased bone resorption activity, in addition to the decrease in bone formation.

### EXAMPLE 8

### CTHRC1 Negatively Regulates Osteoclastogenesis

Based on the expression data (FIG. 1), CTHRC1 is expressed in osteoblasts and osteocytes. The significant upregulation of bone resorption in Cthrc1 null mice suggests that osteoblast- and osteocyte-derived CTHRC1 may function as a key factor negatively regulating osteoclastogenic differentiation. To answer this question, in vitro osteoclastogenic differentiation assays using bone marrow-derived monocytes isolated from Cthrc1 null mice were carried out. This approach avoided any potential effect of endogenous CTHRC1 produced by bone marrow cells. As shown in FIG. 4A and B, bone marrow monocytes are able to differentiate into multinucleated osteoclast in the presence of M-CSF and RANKL and this process is strongly inhibited in the presence of hCTHRC1 as demonstrated by fewer TRAP⁺ osteoclasts (FIG. 4C). Of note, not only was the number of TRAP⁺ osteoclasts decreased, the cells were also significantly smaller in size compared to control treated cells. As a measure of function, the bone resorption capacity of osteoclasts was also analyzed in vitro (FIG. 4D-F). The percentage of resorbed surface area was 63.8%±1.7% for osteoclast cultures supplemented with control-LacZ condition-medium, whereas in the presence of CTHRC1 condition-medium the resorbed surface area was significantly reduced to (46.9%±4.8%, FIG. 4F). Taken together, these data suggest that CTHRC1 derived from bone forming cells may serve as a key factor that negatively regulates osteoclast function. To support this notion even further, bone marrow monocytes were isolated from both wildtype and Cthrc1 null mice, cultured them in osteoclast differentiation medium and found that inactivation of the Cthrc1 gene had no effect on the osteoclast differentiation potential of bone marrow monocytes (FIG. 4G-I). This finding clearly indicated a role of extrinsic CTHRC1 in the osteoclast differentiation process of monocytes. It was also determined by real-time RT-PCR the induction of genes associated with osteoclasts, such as Trap, Ctsk, Rank, c-Fos, and Pparγ. As shown in FIG. 5A, Trap, Ctsk, and c-Fos expression are significantly downregulated in the presence of CTHRC1 on days 4, 7, and 9 of differentiation. Although osteoblasts and osteocytes are considered major sources of Opg, which functions as a decoy receptor for RANKL, it was recently demonstrated that osteoclasts also express Opg (15). Interestingly, rather than an upregulation it was observed a transient downregulation of Opg at day 2 of differentiation. Expression of Rank and Pparγ were not significantly affected by the presence of CTHRC1, suggesting that early stages of monocyte to macrophage differentiation are not influenced by CTHRC1. It was also determined c-Fos, Rankl, Nfatc1, Rank and Trap gene expression within femurs from wildtype and Cthrc1 null mice. As shown in FIG. 5B, c-Fos, Nfatc1, Trap and Rankl expression are significantly upregulated in Cthrc1 null mice when compared with wildtype mice. Rank expression is similar among strains and Opg expression is downregulated in Cthrc1 null mice. Taken together, these data indicate that CTHRC1 plays a pivotal role in inhibiting osteoclastogenic differentiation and bone resorption.

It was determined that M-CSF-stimulated early downstream signaling transduction by Western blotting. CTHRC1 had no effect on levels of p-ERK1/2, p-JNK1/2, or p-p38 (FIG. 9). To gain additional insight into how CTHRC1 modulates osteoclastogenic differentiation, RAW264.7 cells, a macrophage cell line widely used for osteoclast formation, were utilized. RANKL induced robust osteoclast formation in RAW264.7 cells in the presence of control-CM; however, the presence of hCTHRC1 almost completely abrogated osteoclast differentiation (FIG. 6A-C). This finding is consistent with effects of CTHRC1 seen in osteoclastogenic differentiation of primary bone marrow-derived monocytes (FIG. 4). To further verify that the inhibitory activity of osteoclastogenesis was indeed related to the presence of hCTHRC1 in the added conditioned-medium, hCTHRC1 was removed from the conditioned-medium with monoclonal antibodies followed by removal of the CTHRC1/antibody complexes by protein A chromatography. Depletion of the hCTHRC1-CM with antibodies completely abolished its inhibitory effect on osteoclastogenesis (FIG. 6D). The effectiveness of hCTHRC1 removal from the conditioned medium was further verified by ELISA (FIG. 6E), which showed that nearly all hCTHRC1 had been removed by this approach.

To determine at what stage CTHRC1 inhibits osteoclast differentiation, hCTHRC1 was added to osteoclastogenic cultures of RAW 264.7 cells at various stages of differentiation (FIG. 6B). CTHRC1 potently inhibited osteoclastogenesis when it was added within the first 2 days of initiation of differentiation, while exposure of differentiating cells to CTHRC1 at later stages did not inhibit osteoclastogenesis (FIG. 6A-C).

### EXAMPLE 9

### CTHRC1 Negatively Regulates RANKL-Induced NFκB Signaling Activation

The data presented above establish that CTHRC1 blocks osteoclastogenesis rather than cell proliferation. RANKL plays a key role in the induction of osteoclast differentiation, whereas M-CSF promotes proliferation of osteoclast precursors as well as survival of osteoclasts and their precursors. Upon binding to its receptor RANK, RANKL rapidly activates the NFκB pathway by activating IKK, which phosphorylates inhibitory IκBα thereby targeting it for proteasomal degradation. Loss of IκBα then enables translocation of NFκB from the cytosol to the nucleus to activate transcription of the NFκB dependent target genes. Simultaneously, RANKL activates MAPKs, including ERK, p38, and JNK. Moreover, RANKL upregulates the osteoclastogenic transcription factor, NFATC1, and a number of osteoclast proteins, such as Cathepsin K (CTSK) and β3 integrin.

To test whether CTHRC1 inhibits osteoclastogenesis via modulation of RANKL signal transduction, it was examined whether its downstream signaling activation is affected by CTHRC1. It was observed that hCTHRC1 inhibits RANKL-induced phosphorylation and thus degradation of IκBα, resulting in reduced phosphorylation of NFκB (p65) at serine 536 (p-p65), which serves as a readout for NFκB signaling activation (FIG. 6F with quantification shown in FIG. 6H-I). Reduction in p-IκBα levels in the presence of hCTHRC1 (FIG. 6F, H) raises the possibility that CTHRC1 may be inhibiting IKKβ activity, thereby preventing phosphorylation of IκBα. The potential effects of CTHRC1 on transcriptional activity of NFκB were assessed in RAW264.7 cells transfected with a NFκB luciferase reporter and stimulated with RANKL in the presence of hCTHRC1. As shown in FIG. 6J, hCTHRC1-CM completely blocked NFκB reporter activation in response to stimulation by RANKL. These data indicate that CTHRC1 inhibits RANKL-induced NFκB signaling activation by blocking phosphorylation and degradation of IκBα. Finding lower levels of IκBα in femurs of Cthrc1 null mice (FIG. 6K) is consistent with increased NFκB signaling in the mutants. Expression of a constitutively active form of IKKβ (ca-IKKβ) completely reversed the inhibition of osteoclast differentiation by hCTHRC1, confirming that CTHRC1 inihibits NFκB signaling by inhibiting IKKβ activity (FIG. 6L, M).

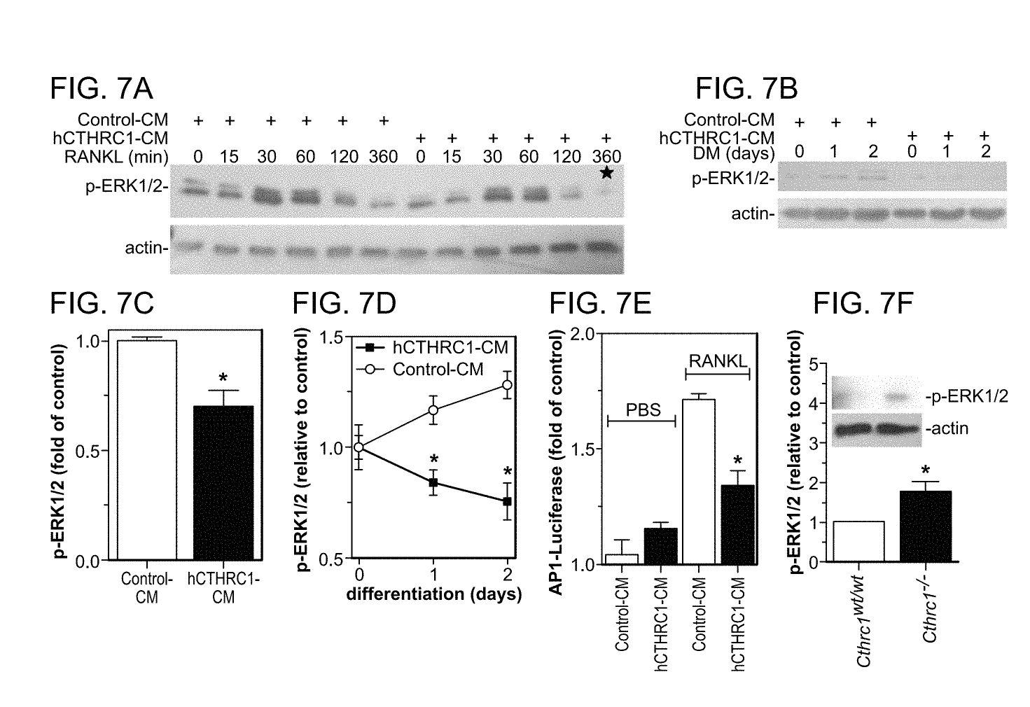

CTHRC1 did not affect early ERK1/2 activation in response to stimulation by RANKL (FIG. 7A). However, levels of activated ERK1/2 (p-ERK1/2) were significantly lower at later stages of differentiation in the presence of hCTHRC1 as seen in FIG. 7A at 360 min and FIG. 7B with quantification shown in FIG. 7C and FIG. 7D, respectively. Consistent with inhibition of ERK1/2 activation by hCTHRC1 is the finding of reduced AP-1 luciferase reporter activity in RAW264.7 cells stimulated with RANKL in the presence of hCTHRC1 (FIG. 7E). Western blotting also revealed elevated p-ERK1/2 levels in femur lysates from Cthrc1 null mice compared to wildtypes (FIG. 7F).

### EXAMPLE 10

### Collagen Antibody-Induced Arthritis is Severely Exacerbated in the Absence of CTHRC1

The NFκB signaling pathway is an important pathway in inflammation. The inhibitory effects of CTHRC1 on NFκB signaling in osteoclasts and macrophage-like RAW264.7 cells prompted us to investigate the role of CTHRC1 in a collagen II antibody-induced arthritis model. Abundant expression of CTHRC1 was recently documented in activated fibroblast-like synoviocytes (FLS) forming the pannus (16). Cells eroding the bone surface show the highest levels of CTHRC1 expression (FIG. 8A). Scoring of arthritis in knee joints and ankle joints as well as quantification of pannus and eroded bone surface revealed vastly increased inflammation in Cthrc1 null joints (FIG. 8D, F, G, H) compared to wildtypes (FIG. 8C, E, G, H).

### REFERENCES CITED

1. Pyagay P, Heroult M, Wang Q, Lehnert W, Belden J, Liaw L, Friesel RE, Lindner V 2005 Collagen triple helix repeat containing 1, a novel secreted protein in injured and diseased arteries, inhibits collagen expression and promotes cell migration. Circ Res 96(2):261-8.
2. Duarte C W, Stohn J P, Wang Q, Emery I F, Prueser A, Lindner V 2014 Elevated plasma levels of the pituitary hormone cthrc 1 in individuals with red hair but not in patients with solid tumors. PLoS One 9(6):e100449.
3. Stohn J P, Perreault N G, Wang Q, Liaw L, Lindner V 2012 Cthrc1, a novel circulating hormone regulating metabolism. PLoS One 7(10):e47142.
4. Kimura H, Kwan K M, Zhang Z, Deng J M, Darnay B G, Behringer R R, Nakamura T, de Crombrugghe B, Akiyama H 2008 Cthrc1 is a positive regulator of osteoblastic bone formation. PLoS ONE 3(9):e3174.
5. Stohn J P, Wang Q, Siviski M E, Kennedy K, Jin Y R, Kacer D, DeMambro V, Liaw L, Vary C P, Rosen C J, Prudovsky I, Lindner V 2015 Cthrc1 controls adipose tissue formation, body composition, and physical activity. Obesity (Silver Spring) 23(8):1633-1642.
6. Takeshita S, Fumoto T, Matsuoka K, Park K A, Aburatani H, Kato S, Ito M, Ikeda K 2013 Osteoclast-secreted CTHRC1 in the coupling of bone resorption to formation. J Clin Invest 123(9):3914-24.
7. Yang W, Wang J, Moore D C, Liang H, Dooner M, Wu Q, Terek R, Chen Q, Ehrlich M G, Quesenberry P J, Neel B G 2013 Ptpn11 deletion in a novel progenitor causes metachondromatosis by inducing hedgehog signalling. Nature 499(7459):491-5.
8. Rowe G C, Vialou V, Sato K, Saito H, Yin M, Green T A, Lotinun S, Kveiborg M, Home W C, Nestler E J, Baron R 2012 Energy expenditure and bone formation share a common sensitivity to AP-1 transcription in the hypothalamus. J Bone Miner Res 27(8):1649-58.
9. Zalli D, Neff L, Nagano K, Shin N Y, Witke W, Gori F, Baron R 2016 The Actin-Binding Protein Cofilin and Its Interaction With Cortactin Are Required for Podosome Patterning in Osteoclasts and Bone Resorption In Vivo and In Vitro. J Bone Miner Res 31(9):1701-12.
10. Bouxsein M L, Boyd S K, Christiansen B A, Guldberg R E, Jepsen K J, Muller R 2010 Guidelines for assessment of bone microstructure in rodents using micro-computed tomography. J Bone Miner Res 25(7):1468-86.
11. Dempster D W, Compston J E, Drezner M K, Glorieux F H, Kanis J A, Malluche H, Meunier P J, Ott S M, Recker R R, Parfitt A M 2013 Standardized nomenclature, symbols, and units for bone histomorphometry: a 2012 update of the report of the ASBMR Histomorphometry Nomenclature Committee. J Bone Miner Res 28(1):2-17.
12. Spatz J M, Ellman R, Cloutier A M, Louis L, van Vliet M, Suva L J, Dwyer D, Stolina M, Ke H Z, Bouxsein M L 2013 Sclerostin antibody inhibits skeletal deterioration due to reduced mechanical loading. J Bone Miner Res 28(4):865-74.
13. Kawai M, Green C B, Lecka-Czernik B, Douris N, Gilbert M R, Kojima S, Ackert-Bicknell C, Garg N, Horowitz M C, Adamo M L, Clemmons D R, Rosen C J 2010 A circadian-regulated gene, Nocturnin, promotes adipogenesis by stimulating PPAR-gamma nuclear translocation. Proc Natl Acad Sci U S A 107(23):10508-13.
14. DeMambro V E, Maile L, Wai C, Kawai M, Cascella T, Rosen C J, Clemmons D 2012 Insulin-like growth factor-binding protein-2 is required for osteoclast differentiation. J Bone Miner Res 27(2):390-400.
15. Kang J H, Ko H M, Moon J S, Yoo H I, Jung J Y, Kim M S, Koh J T, Kim W J, Kim S H 2014 Osteoprotegerin expressed by osteoclasts: an autoregulator of osteoclastogenesis. J Dent Res 93(11):1116-23.
16. Shekhani M T, Forde T S, Adilbayeva A, Ramez M, Myngbay A, Bexeitov Y, Lindner V, Adarichev V A 2016 Collagen triple helix repeat containing 1 is a new promigratory marker of arthritic pannus. Arthritis Res Ther 18:171.
17. Kim D, Mebius R E, MacMicking J D, Jung S, Cupedo T, Castellanos Y, Rho J, Wong B R, Josien R, Kim N, Rennert P D, Choi Y 2000 Regulation of peripheral lymph node genesis by the tumor necrosis factor family member TRANCE. J Exp Med 192(10):1467-78.
18. Kong Y Y, Yoshida H, Sarosi I, Tan H L, Timms E, Capparelli C, Morony S, Oliveira-dos-Santos A J, Van G, Itie A, Khoo W, Wakeham A, Dunstan C R, Lacey D L, Mak T W, Boyle W J, Penninger J M 1999 OPGL is a key regulator of osteoclastogenesis, lymphocyte development and lymph-node organogenesis. Nature 397(6717):315-23.
19. You L D, Weinbaum S, Cowin S C, Schaffler M B 2004 Ultrastructure of the osteocyte process and its pericellular matrix. Anat Rec A Discov Mol Cell Evol Biol 278(2):505-13.
20. Winkeler C L, Kladney R D, Maggi L B, Jr., Weber J D 2012 Cathepsin K-Cre causes unexpected germline deletion of genes in mice. PLoS One 7(7):e42005.
21. Chen J, Shi Y, Regan J, Karuppaiah K, Ornitz D M, Long F 2014 Osx-Cre targets multiple cell types besides osteoblast lineage in postnatal mice. PLoS One 9(1):e85161.
22. Fazeli P K, Horowitz M C, MacDougald O A, Scheller E L, Rodeheffer M S, Rosen C J, Klibanski A 2013 Marrow fat and bone—new perspectives. J Clin Endocrinol Metab 98(3):935-45.
23. Kawai M, Devlin M J, Rosen C J 2009 Fat targets for skeletal health. Nat Rev Rheumatol 5(7):365-72.
24. Rosen C J, Klibanski A 2009 Bone, fat, and body composition: evolving concepts in the pathogenesis of osteoporosis. Am J Med 122(5):409-14.
25. Sims N A, Martin T J 2014 Coupling the activities of bone formation and resorption: a multitude of signals within the basic multicellular unit. Bonekey Rep 3:481.
26. Boyle W J, Simonet W S, Lacey D L 2003 Osteoclast differentiation and activation. Nature 423(6937):337-42.
27. Yamamoto S, Nishimura O, Misaki K, Nishita M, Minami Y, Yonemura S, Tarui H, Sasaki H 2008 Cthrc1 selectively activates the planar cell polarity pathway of Wnt signaling by stabilizing the Wnt-receptor complex. Dev Cell 15(1):23-36.
28. Maeda K, Kobayashi Y, Udagawa N, Uehara S, Ishihara A, Mizoguchi T, Kikuchi Y, Takada I, Kato S, Kani S, Nishita M, Marumo K, Martin T J, Minami Y, Takahashi N 2012 Wnt5a-Ror2 signaling between osteoblast-lineage cells and osteoclast precursors enhances osteoclastogenesis. Nat Med 18(3):405-12.
29. Lee N K, Choi H K, Kim D K, Lee S Y 2006 Rac1 GTPase regulates osteoclast differentiation through TRANCE-induced NF-kappa B activation. Mol Cell Biochem 281(1-2):55-61.
30. Vives V, Laurin M, Cres G, Larrousse P, Morichaud Z, Noel D, Cote J F, Blangy A 2011 The Rac1 exchange factor DockS is essential for bone resorption by osteoclasts. J Bone Miner Res 26(5):1099-110.
31. Mizoguchi F, Murakami Y, Saito T, Miyasaka N, Kohsaka H 2013 miR-31 controls osteoclast formation and bone resorption by targeting RhoA. Arthritis Res Ther 15(5):R102.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of treating or preventing bone loss, low bone mass or a low bone mass-associated condition in a subject, the method comprising:
identifying a subject having or at risk of developing low bone mass or a low bone mass-associated condition;
inhibiting osteoclast differentiation in the subject, thereby treating or preventing bone loss, low bone mass or a low bone mass-associated condition in the subject.

2. The method of claim 1, wherein osteoclast differentiation in the subject is inhibited by administering to the subject an effective amount of a Cthrc1 polypeptide or a Cthrc1 receptor agonist.

3. The method of claim 1, wherein the bone loss, low bone mass or low bone mass-associated condition comprises osteoporosis or arthritis.

4. The method of claim 1, wherein the composition is administered systemically.

5. The method of claim 1, wherein the effective amount of Cthrc1 polypeptide is sufficient to inhibit bone loss by at least 5%.

6. The method of claim 1, wherein the effective amount of Cthrc1 polypeptide is sufficient to increase bone mass by at least 5%.

7. The method of claim 1, wherein the Cthrc1 comprises recombinant Cthrc1.

8. A method of treating or preventing inflammation or an inflammation-associated condition in a subject, the method comprising:
identifying a subject having or at risk of developing inflammation or an inflammation-associated condition;
administering to the subject an effective amount of a Cthrc1 polypeptide or a Cthrc1 receptor agonist, thereby treating or preventing inflammation or an inflammation-associated condition in a subject.

9. A method of inhibiting osteoclast differentiation and activity comprising administering to the subject an effective amount of a Cthrc 1 polypeptide or a Cthrc 1 receptor agonist, thereby inhibiting osteoclast differentiation and activity.

10. The method of claim 9, wherein osteoclast differentiation and activity is decreased by at least 5%.


SEQUENCE LISTING (23 sequences)

SEQ ID NO: 1 - protein, 243 aa, Homo sapiens [sequence omitted]

SEQ ID NO: 2 - DNA, 1279 bp, Homo sapiens
gggagggagagaggcgcgcgggtgaaaggcgcattgatgcagcctgcggcggcctcggagcgcggcggagccagacgctgaccacgttcctctcctcggtctcctccgcctccagctccgcgctgcccggcagccgggagccatgcgaccccagggccccgccgcctccccgcagcggctccgcggcctcctgctgctcctgctgctgcagctgcccgcgccgtcgagcgcctctgagatccccaaggggaagcaaaaggcgcagctccggcagagggaggtggtggacctgtataatggaatgtgcttacaagggccagcaggagtgcctggtcgagacgggagccctggggccaatggcattccgggtacacctgggatcccaggtcgggatggattcaaaggagaaaagggggaatgtctgagggaaagctttgaggagtcctggacacccaactacaagcagtgttcatggagttcattgaattatggcatagatcttgggaaaattgcggagtgtacatttacaaagatgcgttcaaatagtgctctaagagttttgttcagtggctcacttcggctaaaatgcagaaatgcatgctgtcagcgttggtatttcacattcaatggagctgaatgttcaggacctcttcccattgaagctataatttatttggaccaaggaagccctgaaatgaattcaacaattaatattcatcgcacttcttctgtggaaggactttgtgaaggaattggtgctggattagtggatgttgctatctgggttggtacttgttcagattacccaaaaggagatgcttctactggatggaattcagtttctcgcatcattattgaagaactaccaaaataaatgctttaattttcatttgctacctctttttttattatgccttggaatggttcacttaaatgacattttaaataagtttatgtatacatctgaatgaaaagcaaagctaaatatgtttacagaccaaagtgtgatttcacactgtttttaaatctagcattattcattttgcttcaatcaaaagtggtttcaatattttttttagttggttagaatactttcttcatagtcacattctctcaacctataatttggaatattgttgtggtcttttgttttttctcttagtatagcatttttaaaaaaatataaaagctaccaatctttgtacaatttgtaaatgttaagaattttttttatatctgttaaataaaaattatttccaacaaccttaatatctttaaa

SEQ ID NO: 3 - DNA, 1214 bp, Homo sapiens
agaaggtttaaggccggaaagggaaatgaaggggcccggcgctaaccctctaaggacctgttttgcttctgtttaaaccaaatgggcagtctgtcattacacacaccctgggtcttcatatgtggccgccaggtaggagcatcacagtcaagctacgggagaaaacagtttccaggaaactggaaatgaacggcccgagtgctttccaggggctcatctgtgggaagtataatggaatgtgcttacaagggccagcaggagtgcctggtcgagacgggagccctggggccaatggcattccgggtacacctgggatcccaggtcgggatggattcaaaggagaaaagggggaatgtctgagggaaagctttgaggagtcctggacacccaactacaagcagtgttcatggagttcattgaattatggcatagatcttgggaaaattgcggagtgtacatttacaaagatgcgttcaaatagtgctctaagagttttgttcagtggctcacttcggctaaaatgcagaaatgcatgctgtcagcgttggtatttcacattcaatggagctgaatgttcaggacctcttcccattgaagctataatttatttggaccaaggaagccctgaaatgaattcaacaattaatattcatcgcacttcttctgtggaaggactttgtgaaggaattggtgctggattagtggatgttgctatctgggttggtacttgttcagattacccaaaaggagatgcttctactggatggaattcagtttctcgcatcattattgaagaactaccaaaataaatgctttaattttcatttgctacctctttttttattatgccttggaatggttcacttaaatgacattttaaataagtttatgtatacatctgaatgaaaagcaaagctaaatatgtttacagaccaaagtgtgatttcacactgtttttaaatctagcattattcattttgcttcaatcaaaagtggtttcaatattttttttagttggttagaatactttcttcatagtcacattctctcaacctataatttggaatattgttgtggtcttttgttttttctcttagtatagcatttttaaaaaaatataaaagctaccaatctttgtacaatttgtaaatgttaagaattttttttatatctgttaaataaaaattatttccaacaaccttaatatctttaaa

SEQ ID NO: 4 - DNA, 23 bp, Artificial Sequence
  Nfatc1 Forward
tcatcctgtccaacaccaaagtc

SEQ ID NO: 5 - DNA, 22 bp, Artificial Sequence
  Nfatc1 Reverse
atgtgaactcggaagaccagcc

SEQ ID NO: 6 - DNA, 24 bp, Artificial Sequence
  Ctsk Forward
ggcatctttccagttttacagcag

SEQ ID NO: 7 - DNA, 21 bp, Artificial Sequence
  Ctsk Reverse
ggcgttgttcttattccgagc

SEQ ID NO: 8 - DNA, 22 bp, Artificial Sequence
  c-Fos Forward
cgaagggaacggaataagatgg

SEQ ID NO: 9 - DNA, 22 bp, Artificial Sequence
  c-Fos Reverse
agacctccagtcaaatccaggg

SEQ ID NO: 10 - DNA, 22 bp, Artificial Sequence
  Trap Forward
ttccaggagacctttgaggacg

SEQ ID NO: 11 - DNA, 22 bp, Artificial Sequence
  Trap Reverse
gagttgccacacagcatcactg

SEQ ID NO: 12 - DNA, 23 bp, Artificial Sequence
  Opg Forward
gcacagtgaggaggaagacattg

SEQ ID NO: 13 - DNA, 23 bp, Artificial Sequence
  Opg Reverse
acctgagaagaacccatctggac

SEQ ID NO: 14 - DNA, 22 bp, Artificial Sequence
  Rankl Forward
acacctcaccatcaatgctgcc

SEQ ID NO: 15 - DNA, 21 bp, Artificial Sequence
  Rankl Reverse
ttcgtgctccctcctttcatc

SEQ ID NO: 16 - DNA, 24 bp, Artificial Sequence
  Pparg Forward
tttcagaagtgccttgctgtgggg

SEQ ID NO: 17 - DNA, 24 bp, Artificial Sequence
  Pparg Reverse
gatttgtccgttgtctttcctgtc

SEQ ID NO: 18 - DNA, 24 bp, Artificial Sequence
  Rank Forward
catcgttctgctcctcttcatctc

SEQ ID NO: 19 - DNA, 24 bp, Artificial Sequence
  Rank Reverse
cttcacacacttcttgctgactgg

SEQ ID NO: 20 - DNA, 19 bp, Artificial Sequence
  Gapdh Forward
acacattgggggtaggaac

SEQ ID NO: 21 - DNA, 20 bp, Artificial Sequence
  Gapdh Reverse
aactttggcattgtggaagg

SEQ ID NO: 22 - DNA, 18 bp, Artificial Sequence
  Cthrc1 Forward
ccaggtcgggatggattc

SEQ ID NO: 23 - DNA, 20 bp, Artificial Sequence
  Cthrc1 Reverse
agcgtctcctttggggtaat